(19) United States
(12) Patent Application Publication
(10) Pub. No.: US 2019/0006047 A1
(43) Pub. Date: Jan. 3, 2019

(54) SYSTEMS AND METHODS FOR DATA CAPTURE IN AN OPERATING ROOM

(71) Applicant: Sharp Fluidics LLC, Los Altos Hills, CA (US)

(72) Inventors: Josef E. GOREK, Ross, CA (US); Kenneth B. TRAUNER, San Francisco, CA (US); Douglas G. RIMER, Los Altos Hills, CA (US)

(21) Appl. No.: 15/961,703

(22) Filed: Apr. 24, 2018

Related U.S. Application Data
(63) Continuation of application No. PCT/US16/59589, filed on Oct. 28, 2016.
(60) Provisional application No. 62/248,091, filed on Oct. 29, 2015.

(51) Int. Cl. G16H 40/63; G16H 40/20; G06K 9/00; G06K 9/62; A61B 90/90; A61B 90/00

(52) U.S. Cl. CPC G16H 40/63; G16H 40/20; G06K 9/00355; A61B 2090/0807; A61B 90/90; A61B 90/08; G06K 9/6288

(57) ABSTRACT

The data from several sensors can be measured to provide improved measurement of surgical workflow. The data may comprise times at which needles are removed from suture packs and placed in receptacles. The surgical workflow data may comprise data from several instruments such as removal and placement time of surgical instruments and electrocautery devices. The data from several sensors can indicate vital statistics of a patient or environmental conditions of an operating room. The data from several sensors can indicate the presence, absence, arrival, or departure of one or more actors in a surgical workflow. The data from several sensors can be registered with a common time base and a report generated. The report can indicate a performance of individuals and groups of participants in a surgical workflow.

### CROSS-REFERENCE

This application is a continuation of PCT Application No. PCT/US2016/059589, filed on Oct. 28, 2016, entitled “SYSTEMS AND METHODS FOR DATA CAPTURE IN AN OPERATING ROOM” [Attorney Docket No. 48222-706.601], which claims priority to U.S. Provisional Patent Application Ser. No. 62/248,091, filed on Oct. 29, 2015, entitled “SYSTEMS AND METHODS FOR DATA CAPTURE IN AN OPERATING ROOM” [Attorney Docket No. 48222-706.101], the entire contents of which are incorporated herein by reference.

The subject matter of the present application is related to U.S. application Ser. No. 14/697,050, filed on Apr. 27, 2015, entitled “Systems and Methods for Increased Operating Room Efficiency” [Attorney Docket No 48222-703.201], and PCT/US2015/027659, filed Apr. 24, 2015, entitled “SYSTEMS AND METHODS FOR INCREASED OPERATING ROOM EFFICIENCY” [Attorney Docket No 48222-703.601]; the entire contents of which are incorporated herein by reference.

### BACKGROUND OF THE INVENTION

The use of an operating room can present expensive medical service costs. It is estimated that operating room time can cost between about $30 to $100 per minute. The high costs of operating room use can be at least partially attributed to the cost of each employee's time in the operating room. Therefore, increasing the efficiency of the employees within the operating room can reduce the time for each procedure and thereby the overall cost of the procedure.

During a procedure in an operating room, it can be important to accurately track usage and/or movement of various objects. In particular, it is important to accurately account for small objects such as needles and sponges, which may be at risk of accidentally being left in a patient. Generally, if a needle becomes unaccounted for during the surgery, steps need to be taken to ensure that the needle has not been accidently left in the patient. Accounting for needles during a surgical procedure in an accurate manner can be time-consuming and laborious, often requiring a scrub technician, surgical assistant, or circulating nurse to count unused needles and used needles to ensure that all needles are accounted for. Such a process can not only contribute to a reduction in the efficiency of the workers in the operating room, but also distract assisting personnel in the operating room from being able to fully focus on the needs of the surgeon. Therefore, it would be desirable to provide improved systems and methods for tracking usage of surgical objects such as needles in an operating room.

Prior methods and apparatus for measuring surgical work flow are less than ideal in at least some respects. Although millions of surgeries are performed each year, the data recorded from such surgery is less complete than would be ideal, and many aspects of surgical procedures are undocumented in at least some instances. For example, the tracking of placement times of sharp objects such as needles into needle receptacles can be less than ideal in at least some instances. The counting and reconciliation of needles can be manual and time consuming. Also, the amount of time to close a surgical incision can require more effort than would be ideal.

As operating room time is expensive, surgical work flow that is less than ideal may not be adequately documented. Delays during surgery may not be clearly documented, and performance metrics such as wound closure may not be adequately captured to provide an estimate of performance of surgeons and support staff.

Surgical reports can include less information than would be ideal. For example current surgical reports may contain less information than would be ideal to determine the performance of physicians and staff, and also the profile of the surgery itself can be less than ideal. Also, prior surgical reports may provide less than ideal information for a physician to follow a patient following surgery.

In light of the above, it would be desirable to provide improved methods and systems for data capture in operating rooms. Ideally, such methods and systems would provide improved efficiency, outcomes, and safety.

### SUMMARY OF THE INVENTION

The present invention relates to systems and methods for data capture in an operating room, and in particular, to automated or assisted data capture. Embodiments of the present invention can reduce or eliminate human error (or intentional misreporting) in conventional operating room data capture by directly collecting, sanitizing, and aggregating data from a variety of sensors, and facilitate the capture of previously unreported or analyzed operating room data. In some embodiments, the types of operating room data captured can include a usage of surgical and other instruments in the operating room during an associated surgical procedure. Accordingly, the occurrence of retained foreign objects, such as needles or sponges, may be diminished or eliminated by reconciling instrument use data, for example. The operating room data captured can also include audio, image, and video data related to surgical procedure. Accordingly, surgeons' comments and annotations, interactions between operating room personnel, and critical stages of surgery, etc., may all be saved, replayed, reviewed, cross correlated, tagged, or analyzed for any number of purposes, for example. The operating room data captured may also include personnel data related to the identity, and presence, arrival and exit of various surgical team members from the operating room and/or sterilized zones. Sensors may detect or determine the presence, type, or identity of personnel (and instruments and equipment) in the operating room or other sterile or sub sterile zones. The opening and closing of operating room doors permits the exchange of moisture. Accordingly, vectors of infection may be reduced by limiting ingress and egress of personnel, instruments and equipment to the operating room, for example.

The present invention further relates to systems and methods for analyzing and formatting captured operating room data for presentation to users. Accordingly, decisions may be made by health care administrators and other stakeholders, based on comprehensive, automatically generated reports, on how to more efficiently and effectively staff surgical procedures and manage limited operating room resources. Embodiments of the present invention can aggregate and report operating room data captured from a variety of sources as an organized human-readable workflow according a unified timeline.

The present invention yet further relates to systems and methods for predictive analytics and making automated changes to operating room or surgical team configurations in order to increase efficiency. Embodiments of the present invention can analyze the performance of a surgical team, a surgical team member such as the surgeon, or the performance of pairs or other subsets of operating personnel, generally, or for particular surgical procedures, types of patients, time of day, etc. Moreover, embodiments of the present invention can staff surgical teams to suit a particular surgical procedure or patient, modify an existing surgical team to improve a deficiency of the surgical team, or to increase or maximize an efficiency of limited surgical resources. In some embodiments, surgical procedures and teams may be staffed and adjusted in real time during the actual surgical procedure, for example based on a predicted time of surgical procedure completion, so as to avoid multiple surgical procedures concluding around the same time or prevent a surgical procedure from extending past a closing time of a suite of operating rooms or clinic. Accordingly, the risk of becoming unexpectedly bottlenecked by limited resources such as sterilization teams can be diminished or eliminated.

Specific reference is made herein to capturing data related to the dispensing and securing of needles. Additional embodiments described herein are well suited for capturing other data related to various procedures performed in an operating room, such as the amount of energy used during a medical procedure, the movement of various objects within the operating room, and visual and/or audio recordings of the procedures.

The methods and apparatus disclosed herein provide improved measurement of surgical workflow. The data from several sensors can be measured to provide improved measurement of surgical workflow. The data may comprise times at which needles are removed from suture packs and placed in receptacles. The surgical workflow data may comprise data from several instruments such as removal and placement time of surgical instruments and electrocautery devices. The data from several sensors can be registered with a common time base and a report generated. The report may comprise an interactive report that allows a user to determine additional detail of the surgery.

In a first aspect, the present invention includes an apparatus to measure surgical workflow. In an example embodiment, the apparatus includes a processor, which may be a processor system. The processor may be configured with instructions to receive inputs corresponding to a plurality of surgical parameters related to surgery of a patient. The plurality of inputs may include a plurality of times corresponding to one or more of removal of needles from a suture pack or placement of needles in a needles receptacle. The processor may be configured to provide an alert when a first needle and a second needle have been removed from a suture pack without the first needle having been placed in a needle receptacle or when a suture needle has been removed from a pack before the needle has been placed in a receptacle. The plurality of inputs may include a plurality of times at which each of a plurality of needles is removed from a suture pack or a plurality of times at which each of a plurality of needles is placed in a needle receptacle.

In some embodiments, the plurality of inputs may include a unique identifier from a suture pack; the plurality of inputs may include a plurality of unique identifiers from one or more of a plurality of suture packs or each of a plurality of needles; and/or the plurality of inputs may include a plurality of unique identifiers from a plurality of needle receptacles. The plurality of inputs may include a plurality of unique identifiers from a plurality of suture packs and a plurality of unique identifiers from a plurality of needle receptacles and a plurality of times at which each of the plurality of needles is removed from a corresponding suture pack and a plurality of times at which each of the plurality of needles is placed in a corresponding needle receptacle.

The above alerts, unique identifiers and other features, and methods and apparatus may be used with a system for reconciling needles. In some embodiments, beyond maintaining a conventional needle count, the system can track can track whether a same needle was plucked and returned, whether it was plucked and returned to the particular receptacle associated with the originating suture pack, or whether it was plucked and returned in order (i.e., without intervening needles). Moreover, the plurality of inputs may include a unique identifier of a person wearing a surgical barrier or a unique identifier of a surgical barrier worn by a person during surgery. Accordingly, the system may track if the same or an appropriate person removed and returned a needle. Determining who has interacted with a needle or other surgical instrument can be important where communicable or infectious disease is a factor.

In some embodiments, the processor may include instructions to register the plurality of times with a plurality of times from one or more of an optical image, a physician dictation, a video image, a smartphone image, a fluoroscopy radiation dosage, an x-ray radiation dosage from an x-ray, in instrument removal from a holder, an instrument placement into a holder, an electrocautery dosage from an electrocautery device, an implant time at which an implant is placed in the patient, an audio recording, or an image.

In some embodiments, the processor may include instructions to determine an amount of time to close a surgical incision in response to the plurality of times. The processor may include instructions to generate a graph with a common time base, or according to a unified timeline, for one or more of suture removal from a pack, suture placement in a suture receptacle, a video image, a physician dictation, a video image, a smartphone image, a fluoroscopy radiation dosage, an x-ray radiation dosage from an x-ray, in instrument removal from a holder, an instrument placement into a holder, an electrocautery dosage from an electrocautery device, an implant time at which an implant is placed in the patient, an audio recording, or an image. The graph may include an interactive data file in which a user can identify a structure of the graph and view additional detail of the structure.

The identified structure of the graph may comprise information related to one or more of suture removal from a pack, suture placement in a suture receptacle, a video image, a physician dictation, a video image, a smartphone image, a fluoroscopy radiation dosage, an x-ray radiation dosage from an x-ray, in instrument removal from a holder, an instrument placement into a holder, an electrocautery dosage from an electrocautery device, an implant time at which an implant is placed in the patient, an audio recording, or an image.

In another aspect, the present invention includes a method to measure surgical workflow. In an example embodiment, the method includes receiving processor inputs corresponding to a plurality of surgical parameters related to surgery of a patient. The processor may provide an alert when a suture needle has been removed from a pack before the needle has been placed in a receptacle. The processor may provide an alert when a first needle and a second needle have been removed from a suture pack without the first needle having been placed in a needle receptacle.

The plurality of inputs may comprise a plurality of times corresponding to one or more of removal of needles from a suture pack or placement of needles in a needles receptacle. The plurality of inputs may comprise a plurality of times at which each of a plurality of needles is removed from a suture pack. The plurality of inputs may comprise a plurality of times at which each of a plurality of needles is placed in a needle receptacle. The plurality of inputs may comprise a unique identifier from a suture pack, plurality of unique identifiers from a plurality of suture packs, or a plurality of inputs may comprises a plurality of unique identifiers from a plurality of needle receptacles. The plurality of inputs may comprise a plurality of unique identifiers from a plurality of suture packs and a plurality of unique identifiers from a plurality of needle receptacles and a plurality of times at which each of the plurality of needles is removed from a corresponding suture pack and a plurality of times at which each of the plurality of needles is placed in a corresponding needle receptacle.

The method may include registering the plurality of times with a plurality of times from one or more of an optical image, a physician dictation, a video image, a smartphone image, a fluoroscopy radiation dosage, an x-ray radiation dosage from an x-ray, in instrument removal from a holder, an instrument placement into a holder, an electrocautery dosage from an electrocautery device, an implant time at which an implant is placed in the patient, an audio recording, or an image. The method may include determining an amount of time to close a surgical incision in response to the plurality of times.

The method may include generating a graph with a common time base for one or more of suture removal from a pack, suture placement in a suture receptacle, a video image, a physician dictation, a video image, a smartphone image, a fluoroscopy radiation dosage, an x-ray radiation dosage from an x-ray, in instrument removal from a holder, an instrument placement into a holder, an electrocautery dosage from an electrocautery device, an implant time at which an implant is placed in the patient, an audio recording, or an image The graph may include an interactive data file in which a user can identify a structure of the graph and view additional detail of the structure.

In another aspect, the present invention includes an apparatus. In an example embodiment, the apparatus comprises a display and a processor coupled to the display. The processor may comprise instructions to show a graph indicating a plurality of times corresponding to one or more of removal of needles from a suture pack or placement of needles in a needles receptacle.

In yet another aspect, the present invention includes and apparatus for surgery. In an example embodiment, the apparatus may include a display and a processor. The processor may be coupled to this display and comprise instructions to receive user input to trigger an optical image capture and to store the optical image with a time stamp. The processor may also comprise instructions to receive audio input from a user in response to an audio trigger. The apparatus may include a sterile container. The sterile container may be configured for said user to input instructions through said sterile container.

The display may comprise a touch screen display, e.g., of a smart phone, tablet, or other mobile computing device. The sterile container may be configured for the user to provide input to the touch screen display through the sterile container. The sterile container may comprise a sterile bag. The apparatus may include a camera or microphone. A user-adjustable support may be configured to support one or more of the camera or microphone, the display and the processor in order for a user to position said camera to capture surgical images, video, or audio.

In yet another aspect, the present invention includes a method. According to various example embodiments, the method may provide the apparatus described hereinabove.

Also in an aspect, the present invention includes a method for assigning a surgical team to a surgical procedure. In an example embodiment, the method may include receiving one or more surgical parameters associated with the surgical procedure and selecting one or more members of the surgical team based on the surgical parameters. The method may further include outputting, over a computer network, an indication of the one or more members of the surgical team. The method may further comprise displaying to a user an indication of the one or more members of the surgical team.

The surgical parameters may include at least one of a length of time of surgical procedure, a type of surgical procedure, a complexity of surgical procedure, or a patient receiving the surgical procedure. The one or more members of the surgical team comprises at least one of a surgeon, assistant surgeon, scrub tech, anesthesiologist, anesthesia technician, nurse, or assistant, or any other personnel found in an operating room.

The method may include receiving an indication of a first plurality of members of a surgical team, receiving an indication of a deficiency or point for improvement of the surgical team, and modifying based on the deficiency, the surgical team to comprise a second plurality of members. Modifying may mean adding, subtracting, or substituting personnel from the surgical team. The deficiency may be determined by a user, or programmatically determined by a processor of an example system of the present invention. The deficiency may be identified based on analyzing a past performance of the surgical team, either collectively, or individually. The deficiency may be related to one or more of a level of skill, level of experience, speed, cost, number of team members, team chemistry, scheduling, or fatigue level of the surgical team or its team members. The selected or modified surgical team may be assigned to an operating room to complete a corresponding surgical procedure.

In another aspect, the present invention includes a method for assigning surgical teams to an operating room. In an example embodiment, the method may include receiving a plurality of surgical procedures and one or more respective surgical parameters corresponding to each surgical procedure, receiving at least one operating room, receiving a plurality of surgical team members, the plurality of surgical team members including at least one of a surgeon, assistant surgeon, scrub tech, anesthesiologist, anesthesia technician, nurse, or assistant. The method may further include assigning, for each surgical procedure, based on the respective surgical parameters, a corresponding surgical team comprising a subset of the plurality of surgical team members to the surgical procedure.

The surgical procedure and surgical team may be assigned to an operating room to complete the surgical procedure. The assignment of surgical procedures and surgical teams to operating rooms may be based on the available operating rooms and their configurations (e.g., size, equipment, etc.). Accordingly, the method may also include estimating a length of time to complete each surgical procedure, for example based on the complexity of the procedure and the (track record or predicted performance of the) respective surgical team assigned, and assigning operating rooms based on the length of the procedures. The operating rooms, and surgical teams, may also be assigned based on other factors such as rate of operating room turnover, operating room cost (e.g., as a function of time), surgical team skill, a fatigue level of the surgical team/members, legally or work place mandated break time, etc.

When multiple operating rooms are available, for example, the method may include assigning a particular personnel member, for example a surgeon, to multiple surgical teams or surgical procedures that overlap in time. Accordingly, the surgeon may travel from a first operating room to a second operating room while a surgical procedure in the first operating room is ongoing. The method may include assigning personnel to multiple surgical procedures based on critical stages associated with the surgical procedures, for example so that a surgeon may be present for the critical stages of two overlapping surgical procedures. A critical stage of a surgical procedure may be based on or correlated with critical decision making and a high level of surgical risk. In some implementations the critical stages of a surgical procedure are input by a user or preprogrammed. In another embodiment, the critical stages of a surgical procedure may be programmatically determined by systems of the present invention, for example based on data from previous surgical procedures. Critical stages may be determined based on recognizing patterns of instrument use from the previous surgical procedures, for example, that were associated with critical stages (human input or programmatically determined) of the previous surgical procedures, or based on recognizing patterns of surgeon movement or gesture from the previous surgical procedures. In some implementations, machine learning, in particular, deep learning, can be applied to the problem of programmatically determining critical stages of a surgical procedure, and indeed many other programmatic determinations described herein.

The surgical team member selection and assignment to surgical procedures and operating rooms may be output or communicated over a computer network or displayed to a user. In some embodiments, the assignment of surgical procedures to operating rooms may be reported in a Gantt-style chart. The chart may be updated in real time during the day to reflect the deviation of the actual use of the operating rooms from the initial schedule, and to show updated estimates for surgical procedures scheduled for the day that have yet to start or conclude.

In yet another aspect, the present invention includes a method for assessing performance of a surgeon or other operating room personnel. The method may include receiving surgical data related to one or more past surgical procedures the surgeon participated in, and determining the performance of the surgeon or other personnel based on the surgical data. The method may include outputting or displaying an indication of the performance of the surgeon. In some embodiments, the indication of the performance of the surgeon, or the analysis itself of performance of the surgeon may be related to a limited number of portions or stages of various surgical procedures. For example, a surgeon (or other personnel) may be graded according to the following time periods: i) time a patient is admitted to an operating room until time of incision; ii) time of incision until time surgical procedure has ended; iii) time of incision closure until patient is out of the operating room; or iv) time a previous patient is out of the operating room before a next patient can be admitted to the operating room.

The past surgical data may include various types of data, including suture data, motion data, patient data, surgical procedure data, operating room environment data, etc., related to past surgical procedures. In some embodiments, suture data includes historical data related to a number of needles used by the surgeon to close an incision and a length of time when the needless were in use. This can indicate a suturing speed of the surgeon, assistant surgeon, or resident. The suture data may include historical data related to a number of sutures used. The number of sutures used is related to incision length which can be an indicator of morbidity. The suture data may also include a type of suture used or a type of tissue sutured, for example to permit more accurate comparisons between situations.

Other data may also be used to color or inform a performance of a surgeon (or other surgical personnel), such as patient data related to past surgical procedures.

The past surgical data may include motion data corresponding to recorded movements or gestures of the surgeon in an operating room during the past surgical procedures. The method may include determining, based on the motion data, a period of waiting or dead time of the surgeon during surgical procedures. Time spent by the surgeon waiting for other actors may not be the fault of the surgeon. Accordingly, this can be a factor in assessing the performance of the surgeon. Thus, the method includes analyzing movement of limbs or hands of the surgeon during the surgical procedure and of other active personnel in the operating room, and also movement of one or more tools used by the surgeon and other actors. The movement of tool may be determined based on one or more of optical recognition, RFID, conductivity, induction, auditory cues, or other technologies or techniques discussed herein. Optical recognition may be based on machine readable codes, color codes, or object recognition and recorded by cameras, scanners, or other image capture devices.

The patient data may include an indication of a body fat level of a patient, for example, at least one of a weight, height, BMI, or body fat percentage of the patient. The patient data may include an age or gender of a patient at the time of the surgical procedure and any skin-related disease or condition of the patient. The patient data may include scar tissue data, medication taken by the patient or medical treatment received by the patient (e.g., chemotherapy). The past surgical data may include surgical procedure data related to past surgical procedures. The surgical procedure data may comprise at least one of a type, complexity, difficulty, success rate, or average procedure length associated with past surgical procedures.

In particular, the determining the performance of scrub tech personnel, may be related to how long a surgeon had to wait to be handed or receive instruments during a past surgical procedure. The performance of the circulating nurse may be related to a frequency or length of time a circulating nurse has to leave and a nature of the items retrieved. The performance of an aesthetician may be related to delays in preparing a patient for surgery, or delays or complications from rotations between aestheticians during past surgical procedures.

In some embodiments, once performance levels are established, useful prediction may be made based off the performance levels in real time to alter the course of a surgical procedure. For example, for a surgical procedure that is determined to be half finished but behind schedule because of poor pre-surgical preparation, an additional circulating nurse may be designated to assist. Real time changes may also be made in the context of surgical-unit wide planning. For example, several surgical procedures may be predicted to end at similar times. Accordingly, there may not be enough sterilization teams to attend to the operating rooms post-surgery without impeding work flow of the surgical unit. Accordingly, one or more of the surgical procedures may have personnel or other changes implemented in real time to avoid the bottleneck.

In another aspect, the present invention includes a method for determining workflow in an operating room related to a surgical procedure, the method comprising recording operating room data related to at least one of, and particularly a combination of: i) a use of instruments in the operating room during the surgical procedure; ii) audio, image, or video in the operating room during the surgical procedure; or iii) personnel in the operating room during the surgical procedure. The method further includes generating a graph or chart based on the operating room data. The graph may be output, e.g., over a computer network, or displayed to a user, e.g., locally. The graph may depict the operating room data as a function of time, in particular mapping multiple types of operating room data to a single timeline. The graph may juxtapose or otherwise display together, sequence of events or timelines constructed from two or more types of operating data. Accordingly, events from multiples types of operating room data may be displayed according to a unified timeline.

Recorded data may be received from sensors and other sources with corresponding timestamps. However, the timestamps may be formatted inconsistently, based on different time bases or time zones, or otherwise off sync. Accordingly, the method may include receiving, sanitizing, and standardizing time-stamp data associated with recorded data from disparate sources. Recorded data may also be received without a timestamp. In some instances, the method may include assigning a timestamp to the data, such as based on when the data was received by the system. Thus, previously un-tagged live data and other data may be temporally oriented with other externally time-stamped data. Un-stamped recorded data may also be assigned a time stamp based on a timestamp of other recorded data that is related to the un-stamped data. For example, a time-indexed video feed can be used to assign a timestamp to sensor data recorded for an event that was visible in the video feed but received from another sensor device in the operating room.

In some embodiments, the graph may be interactive, allowing a user to view events within particular time slices, or mark events between different data types as related. Groups of related events may also be determined programmatically by systems of present invention, for example based on correlations in time or causal relationships between events.

The recording of operating room data related to the use of instruments may include collecting or recording suture pack data (as described elsewhere herein). As with needles, other instruments in the operating room may be tracked, including data related to the movement, opening, use, retiring, sterilization, or disposal of such objects. The number of instruments in use or in the surgical field at any given time during the surgical procedure may be recorded. The recording of operating room data related to the use of instruments may also include monitoring motion of personnel in the operating room, especially in conjunction with motion of tools.

In some embodiments, data related to the use of instruments comprises a flow of energy directed to the patient from one or more instruments in the operating room. The energy may include one or more of x-ray energy, heat energy, laser energy, radio-frequency energy, or ultrasound energy. The instruments may include an electrocautery pen, fluoroscope, x-ray machine, laser, or ultrasound transducer.

In some embodiments, data related to the use of instruments comprises a flow into or total volume in the patient of liquid from one or more instruments in the operating room. The liquid may include one or more blood, plasma, saline, anesthetic agent, pain killer, blood thinners, or antibiotics.

In some embodiments, operating room data related to audio, image, or video may be recording with one or more recording devices. A first device may record continuously throughout a surgical procedure while a second device may start or stop recording in the middle of the procedure. For example, the second recording device may be motion-activated, sound-activated, voice-activated, or activated based on reaching a particular stage of the surgical procedure.

In some embodiments, the operating room data related to personnel in the operating room comprises the presence or absence of personnel in the operating room. The presence or absence of personnel may be tracked by monitoring arrivals to and departures from the operating room. Arrivals and departures may be tracked based on scanning a badge or ID of operating room personnel. The scanning may be based on optical recognition or another technology, for example RFID. In another embodiment, personnel can be required to sign in to the operating room, for example, by presenting biometric verification. In some embodiments, it may be determined whether personnel is dressed properly for a particular zone, e.g., properly scrubbed for a sterile environment.

Arrivals and departures may be associated with a door or other opening to the operating room. Some doors may open to various degrees or amounts depending on whether supplies, people, or large equipment is being moved. Moreover, some doors or entrances are associated with a particular direction (i.e., one way). Embodiments of the present invention are not limited to just maintaining a count of arrivals and departures but tracking which doors are marked for ingress and egress, whether such doors were used appropriately, to what extent the doors were opened, and how opening a door introduced moisture into the operating room.

In another aspect, the present invention includes a method for performing a cleanliness audit of the operating room based on analyzing the operating room data. The method may include capturing a first image of the operating room before the surgical procedure, capturing a second image of the operating room after the surgical procedure, and determining a change in cleanliness of the operating room during the surgical procedure based on comparing the first image to the second image.

The first image may be a “before” image captured preceding the surgical procedure for a before-and-after comparison, or the first image may be a general reference images used a baseline for comparing images captured after various other surgical procedures.

Comparing the first image and second image may include providing a set of reference points in the operating room and analyzing portions of the first image and second image corresponding to the reference points. A set of reference points are changed or rotated between consecutive surgical procedures in a same operating room, or even randomized between surgical procedures. The reference points also may be chosen based on the type of surgical procedure. Reference points may also be chosen based on reviewing the audio, image, or video data, associating events in the audio, image, or video data with one or more locations in the operating room. For example, a location of a spill of blood or body fluids onto the operating room floor may be tagged in a video as reference point for determining whether the operating room was later cleaned effectively.

The performance of a surgical team or a surgical team member may be based on a determination of cleanliness. Leaving dirty operating rooms may increase turnover time and stretch other resources of the surgical unity such as sterilization teams. Relatedly, the cleanliness of sterilization teams may also be evaluated. For example, the method may include

capturing a first image of the operating room after the surgical procedure and before being sterilized, capturing a second image of the operating room after the surgical procedure and after being sterilized, and determining a change in cleanliness of the operating room during the surgical procedure based on comparing the first image to the second image.

Note that beyond the simple image comparison, the cleanliness audit may be also based on identifying one or more surgical instruments or equipment used during the surgical procedure based on the operating room data. These implements may also suitable for or related to reference points.

In some embodiments, the operating room data may include vitals of a patient during the surgical procedure, for example an amount of blood lost by the patient or an amount or urine collected from the patient. Blood loss may be determined by one of the blood loss tracking systems described herein. Urine collection may be determined by one of the urine collection tracking systems described herein. The operating room data may include environmental conditions in the operating room during the surgical procedure, such as temperature, humidity, or light level of the operating room.

In yet another aspect the present invention includes a system for monitoring personnel in an operating room during a surgical procedure. Personnel may include one or more of a surgeon, assistant surgeon, scrub tech, anesthesiologist, anesthesia technician, nurse, assistant, or other actors in the operating room. In an example embodiment, the system includes one or more sensors, a processor, and a memory. The sensor may be related to a scanner. The scanner may be configured to scan at least of one of a badge, RFID, or machine-readable code, biometric signal, or other suitable identifier. The scanner may be positioned in range of an entrance or exit of the operating room or a sterilization barrier or checkpoint, for example, for scrubbing in and out.

The sensor may be related to a camera. The camera and/or processor may be configured to detect personnel or recognize/identify personnel in the operating room. Locations of personnel in the operating room may also be tracked, or just a number of personnel in the operating room or types of personnel in the operating room. The processor may be configured to determine whether certain personnel, e.g., surgeons, are present during certain stages of the surgical procedure, for example one or more critical stages of the surgical procedure. Example stages of the surgical procedure may be as defined elsewhere herein.

The camera may be configured to record a surgical procedure in its entirety, or just a particular portion or stage of the surgical procedure. Where a portion of the surgical procedure is recorded, the camera may be one or more of motion-activated, sound-activated, voice-activated, or activated based on reaching a particular stage of the surgical procedure. The camera and/or processor may be configured to recognize instruments used during the surgical procedure, or movements or gestures of the personnel in the operating room. The processor may determine a stage of the surgical procedure based on the instruments used, or movements or gestures of the personnel. The processor may be configured to determine a dead time associated with the surgical procedure based on the instruments used, or movements or gestures of the personnel.

In still yet a further aspect, the present invention includes a system for mapping operating room flow. In an example embodiment, the system includes a memory, one or more sensors, and a processor. The processor may be configured with instructions to perform methods of the present invention as described herein.

In yet another aspect, the present invention includes a system for tracking urine collected over time by a patient during a surgical procedure. In an example embodiment, the system includes a urine storage vessel, a sensor, a memory, and a processor. The processor may be configured to store the volume of urine collected by the storage vessel at a pre-selected interval, or based on a change in the signal. The sensor may be separate or discrete from the urine storage vessel or integral with the urine storage vessel. The sensor may be related to a pressure transducer disposed between the urine storage vessel and a holder configured to support the urine storage vessel in hanging configuration, or the sensory may be related to a flowmeter disposed at an inlet of the urine storage vessel. The storage vessel may include sensing and control circuitry for determining a volume of urine collected by the storage vessel. The storage vessel may also include a power source for powering the sensing and control circuitry. The system may include a visual display, the processor configured to output an indication of a volume of urine collected by the vessel on the visual display. The indication of the volume of urine may comprise a timestamp.

While embodiments of the present invention are directed to workflow in the operating room, methods, devices, apparatus, systems, and computer-program products of the present invention maybe applicable to data capture in other environments. Various combinations and configurations of the above and other features described herein and contemplated and within the present disclosure.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates a surgical field and a near surgical field, in accordance with embodiments;

FIGS. 2A and 2B illustrate schematic diagrams of exemplary systems for capturing needle usage data, in accordance with embodiments;

FIGS. 3A and 3B schematically illustrate an exemplary system for the electrical sensing of needle dispensing and securing, in accordance with embodiments;

FIGS. 4A-4C illustrate mechanical counter devices that can be used with the needle dispensing unit and/or the needle receptacle to facilitate needle counting, in accordance with embodiments;

FIG. 5 illustrates an optical counter mechanism that can be used with the needle receptacle to indicate the number of stored needles, in accordance with embodiments;

FIG. 6 illustrates an embodiment of the system which can detect the number of needles in the secure zone of the needle receptacle based upon pressure measurements detected by transducers, in accordance with embodiments;

FIG. 7 illustrates how cameras can be used to detect the number of needles that move into the secure zone of the needle receptacle, in accordance with embodiments;

FIG. 8 illustrates other components that can be used with the needle receptacle to perform needle counting, in accordance with embodiments;

FIGS. 9A-9C illustrate an overview of needle tracking, in which the dispensing and securing of needles is reconciled, in accordance with embodiments;

FIGS. 10A and 10B show volar and dorsal views, respectively, of a forearm-mounted barrier, in accordance with embodiments;

FIG. 11 illustrates an overview of the data tracking enabled by the use of the systems and devices disclosed herein, in accordance with embodiments;

FIG. 12 shows a graphical representation of data that may be recorded during a surgical procedure, in accordance with embodiments;

FIG. 13 illustrates a schematic diagram of an exemplary system for surgical workflow monitoring, in accordance with embodiments;

FIG. 14 illustrates a surgical workflow monitoring method, according to embodiments;

FIG. 15 illustrates a surgery design configuration, in accordance with an example embodiment.

FIGS. 16A-16D schematically illustrate exemplary embodiments of urine storage systems for the tracking of urine volume from a patient through time, in accordance with embodiments;

FIG. 17 schematically illustrates an example of the personnel involved in an operating room procedure, in accordance with embodiments;

FIG. 18 illustrates an exemplary work flow for an operating room procedure through time.

FIG. 19 illustrates a schematic diagram of an exemplary system for surgical workflow monitoring, in accordance with embodiments;

FIG. 20 shows a graphical representation of data that may be recorded during a surgical procedure, in accordance with embodiments;

FIGS. 21A-21C and corresponding FIGS. 21A1-21C1 illustrate operating room personnel performing surgical procedures in more than one operating room, in accordance with embodiments;

FIG. 22 illustrates an exemplary digital processing device, in accordance with embodiments; and

FIG. 23 shows a graphical representation of operating room status, in accordance with embodiments.

### DETAILED DESCRIPTION OF THE INVENTION

Described herein are systems and methods for tracking the usage of various surgical objects in an operating room throughout the course of an operating room procedure. Also described herein are systems and methods for capturing the data related to the usage of the various surgical objects throughout the course of the procedure. In particular, systems and methods are disclosed herein for tracking needle usage and capturing needle usage data throughout the course of a procedure. The systems and methods described can provide accurate tracking of the dispensing of unused, sterile needles and the securing of dispensed needles, such that all of the dispensed needles within the operating room can automatically be accounted for. The systems described herein can also be configured to capture data related to the dispensing and securing of needles over the course of the procedure, and store the needle usage data for later review.

The present methods and apparatus can be configured to capture the data related to the use of the surgical objects throughout the course of a surgical procedure. For example, it would be desirable to provide systems and methods for capturing data related to needle usage or the usage of energy by various surgical tools over the course of a surgical procedure. Such captured data can provide a “map” of what happened during the procedure, potentially providing valuable insights regarding how efficiently various steps of the procedure were performed, whether there were any aberrations in any parts of the procedure, etc.

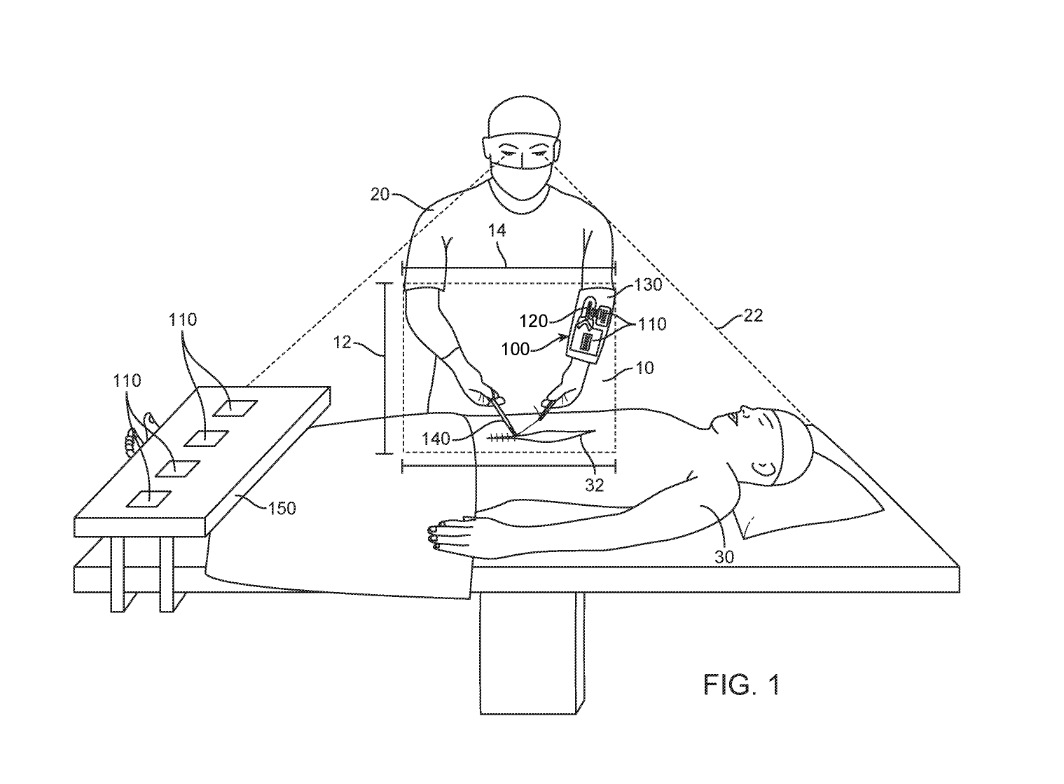

FIG. 1 illustrates a surgeon performing an operation within a near surgical field, using methods and systems in accordance with embodiments. The surgeon of FIG. 1 is shown holding a needle driver with his dominant right hand, while holding a tissue forceps with his non-dominant left hand. A needle tracking system 100 in accordance with embodiments is shown mounted on the surgeon's non-dominant left forearm. The needle tracking system 100 can comprise a needle dispensing unit 110, such as one or more suture packs, and a needle receptacle 120. Each of the dispensing unit 110, the needle receptacle and the barrier 130 may comprise a unique identification. The unique identification can be provided in many ways, and may comprise one or more of a bar code, a quick response (QR) code, and a RFID or color code, for example. In addition, the system may comprise one or more sensors (not shown) configured to sense the dispensing of a needle from the needle dispensing unit and the securing of a needle within the needle receptacle. The system may further comprise a processor (not shown) in communication with the one or more sensors, such that the sensors can transmit to the processor in real time data relating to the movement of needles within the surgical field and more particular within the near surgical field. The processor can be configured to automatically account for every needle in the surgical field, by tracking and comparing the number of dispensed needles and the number of secured needles, as described in further detail herein.

As shown in FIG. 1, the needle tracking system 100 may be arranged within the near surgical field 10 such that the system can track the dispensing and securing of needles by the surgeon 20 within the near surgical field. A “surgical field” can include a space within an operating room where the patient and surgeon are located during surgery. A “near surgical field” 10 can be a much smaller space that is in close proximity to the incision 32 on the patient 30 and the surgeon. The near surgical field 10 may comprise a space disposed between the surgeon 20 and the incision 32. For example, the near surgical field can comprise a length 12 extending between a surgeon and an incision of a patient and a width 14 extending transverse to the length, the width comprising no more than about 24 inches (61 cm) across. The entire near surgical field can also be within the field of view 22 of the surgeon 20.

The embodiments described herein can enable automatic tracking and accounting for needles in the near surgical field, without requiring assistant personnel to count the needles as individual needles are passed in and out of the near surgical field. As shown, the needle tracking system 100 can be supported within the near surgical field so as to allow the surgeon or other user to dispense and secure needles without assistance from another person. For example, the needle tracking system may be supported on a surgeon's non-dominant limb as shown in FIG. 1, so that the surgeon may dispense and secure needles using his dominant hand, without requiring an assistant surgeon or scrub technician to pass individual needles. The needle tracking system may be provided on a support 130 mounted on the volar forearm of a surgeon as shown in FIG. 1. The support 130, which may comprise a puncture-resistant barrier material to help prevent needle stick injuries, can support the needle dispensing unit 110 and the needle receptacle 120, as well as various surgical tools to be used throughout the procedure. Optionally, the support may additionally support the one or more sensors configured to detect needle movement out of the dispensing unit and into the receptacle. The processor configured to receive needle usage data and track needle count may also be coupled to the support, or the processor may be disposed elsewhere within or outside the near surgical field.

Further, additional data related to the procedure may be automatically tracked and captured. For example, the use of one or more tools 140, such as surgical tools used by the surgeon, may be tracked via sensors placed on or near the tools, or near storage locations of the tools, as described elsewhere herein. Each tool may comprise a unique identifier (ID) to track the tool as described herein. A tool may comprise an energy-driven tool, such as an electrocautery pen, and energy use by the energy-driven tool may also be captured and stored in real-time. One or more sensors may also be placed on a support platform 150 placed within the surgical field, such as a Mayo stand. For example, one or more additional needle dispensing units 110 may be placed on the support platform for use by the surgeon during the operation, and a sensor coupled to the support platform may be configured to track the movement of the dispensing units 110 onto or away from the support platform. The support platform may also support one or more sensors configured to capture audio, image, or video data of the procedure, as described elsewhere herein.

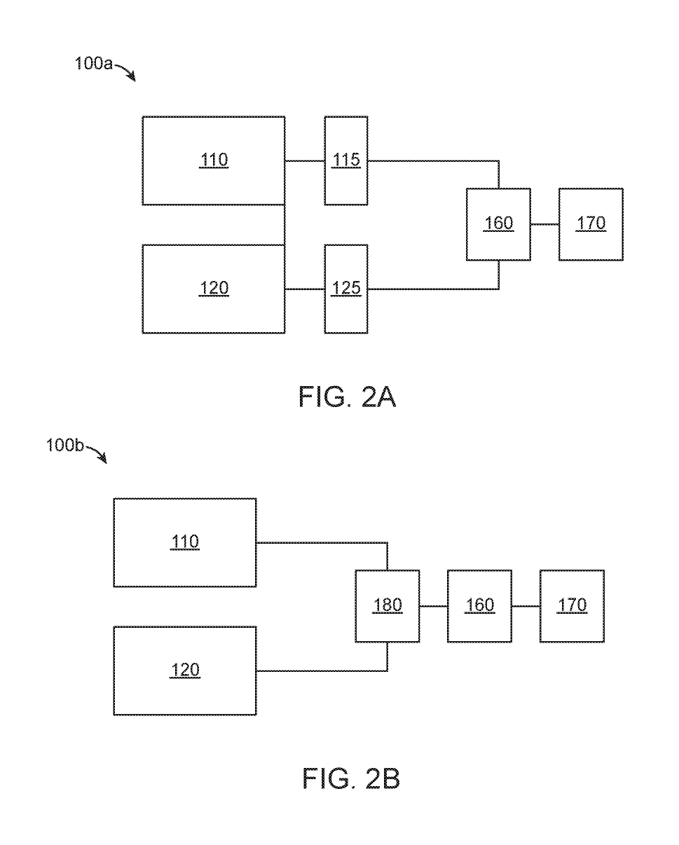

FIG. 2A is a schematic diagram of an exemplary system 100a for capturing needle usage data in accordance with embodiments. The system 100a comprises a needle dispensing unit 110, a needle receptacle 120, and a processor 160 in communication with the needle dispensing unit and the needle receptacle. The needle dispensing unit and needle receptacle may comprise unique identifiers as disclosed herein. The needle dispensing unit may comprise any container configured to store a plurality of sterile needles, such as any commercially available suture needle package, or a custom container of suture needles as described herein. The needle receptacle may comprise any receptacle configured to receive and securely store a plurality of dispensed suture needles, such that the needles stored therein are rendered innocuous and cannot accidentally exit the needle receptacle. The needle dispensing unit is operatively coupled to a sensor 115 configured to sense the dispensing of a sterile needle from the dispensing unit. The needle receptacle is operatively coupled to a sensor 125 configured to sense the securing of a dispensed needle within the needle receptacle. Sensors 115 and 125 may comprise one or more of many types of sensors as described in further detail herein. Sensors 115 and 125 may be the same type of sensor, or they may be different types of sensors. Sensor 115 may be separate from or integrated with the needle dispensing unit. Similarly, sensor 125 may be separate from or integrated with the needle receptacle. Sensors 115 and 125 may be configured to transmit signals to the processor 160 when the dispensing or securing of a needle is detected. The processor 160 can be configured to automatically account for every needle in the surgical field, by tracking and comparing the number of dispensed needles and the number of secured needles, as described in further detail herein. Optionally, the system 100a may further comprise a display 170 in communication with the processor 160, to display the number of dispensed and secured needles or other data related to the tracking of needles as detected by the sensors. Additional circuitry such as wireless communication circuitry can be provided with the dispensing unit to track usage of the needles and to transmit the unique identifiers and time stamp data.

FIG. 2B is a schematic diagram of another exemplary system 100b for capturing needle usage data in accordance with embodiments. The system 100b comprises a needle dispensing unit 110, a needle receptacle 120, and a processor 160 as described in reference to system 100a shown in FIG. 2A. The system 100b further comprises a sensor 180 operatively coupled to both needle dispensing unit 110 and needle receptacle 120. The sensor 180 may be configured to sense the dispensing of a sterile needle from the dispensing unit and the securing of a dispensed needle within the needle receptacle. The sensor 180 can be further configured to transmit signals to the processor 160 when the dispensing or securing of a needle is detected. The processor 160 may optionally be in communication with a display 170 to display the needle tracking data. Additional circuitry such as wireless communication circuitry can be coupled to the processor to track usage of the needles and to transmit the unique identifiers and time stamp data as described herein.

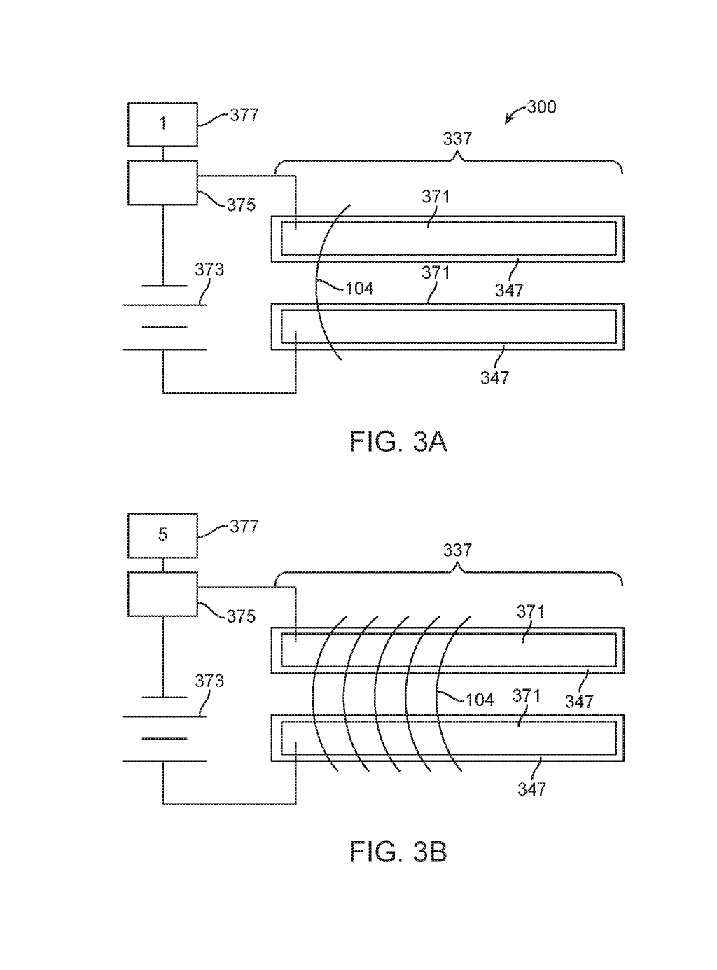

FIGS. 3A and 3B schematically illustrate an exemplary system for the electrical sensing of needle dispensing and securing. Additional circuitry such as wireless communication circuitry can be coupled to the electrical sensing circuitry to track usage of the needles and to transmit the unique identifiers and time stamp data as described herein. The needle tracking system as described herein may comprise a needle dispensing unit and a needle receptacle each having an integrated electrical sensor 300 for detecting the dispensing of needles from the dispensing unit and the securing of needles within the needle receptacle. The electronic sensor can be powered by a battery 373 such as a lithium ion battery or any other suitable electrical power source. The needle dispensing unit or receptacle may comprise conductive elements 371, coupled to an interior surface of the dispensing unit or receptacle so as to contact a needle 104 disposed within the dispensing unit or receptacle. For example, the conductive elements may be adhered to or directly printed onto an inner top or bottom surface of the dispensing unit or receptacle. In embodiments wherein the needle dispensing unit or receptacle comprises compressive members 347 on either side of a needle driver slot, the conductive elements 371 can be mounted on the compressive members 347. The conductive elements 371 can be pressed into physical contact with each needle 104 that is placed in the secure zone 337 by the compressive members 347. The electrical counter mechanism can include control circuitry 375 and a visual display 377 coupled to the control circuitry 375.

The electrical counter mechanism can comprise an electrical circuit with electrical current flowing through the needles 104 in the secure zone and the control circuitry 375. The electrical resistance changes based upon the number of needles 104 stored in the secure zone in contact with both of the conductive elements 371. The electrical circuit can have a higher electrical resistance with fewer needles 104 in the secure zone. The electrical resistance can decrease with more needles 104 in the secure zone. Each of the used needles 104 can each have an electrical resistance between the conductive elements 371 that is substantially the same. Thus, each of the used needles 104 can function as a resistor in the electrical circuit and multiple used needles 104 in the secure zone can function as a plurality of parallel resistors.

The basic electrical circuit equation is V=I R where V is voltage, I is current and Rₜₒₜₐₗ is the cumulative needle resistance. The cumulative electrical resistance can decrease with each additional stored needle in the secure zone. The equation for parallel resistors is 1/Rₜₒₜₐₗ=1/R₁+1/R₂+1/R₃ . . . . However, the resistances of the needles can all be substantially equal, i.e. R₁=R₂=R₃ where R₁ is the electrical resistance of each used needle. The cumulative electrical resistance needles equation becomes 1/Rₜₒₜₐₗ=N/R₁ or Rₜₒₜₐₗ=R₁/N where N=number of needles. Thus, the number of needles can be calculated with the electrical circuit by V=I R₁/N or N=I R₁/V. Changes in the cumulative resistance and impedance of the parallel needles can alter the electrical current flowing through the electrical circuit. The voltage V and R₁ values can be substantially constant. Thus, changes in the electrical current (I) are based upon the number of parallel needles in the secure zone. The control circuitry 375 can include an ammeter that measures the electric current (I) in the circuit and based upon the measured current, the control circuitry 375 can calculate the number of needles in the secure zone. The control circuitry 375 can output a signal to the visual display 377 that corresponds to the number of needles in the secure zone. In an embodiment, the number of needles N can be displayed on the visual display 377. With reference to FIG. 3A the visual display 377 can display the number “1” which corresponds to the single needle 104 between the conductive elements 371. With reference to FIG. 3B, the visual display 377 can display the number “5” which corresponds to the five needles 104 between the conductive elements 371. In other embodiments, the visual display 377 can output any other display that can indicate the number of needles in the secure zone. For example, the display can use individual lights to represent each needle. Each needle in the secure zone can be represented by a single corresponding illuminated light.

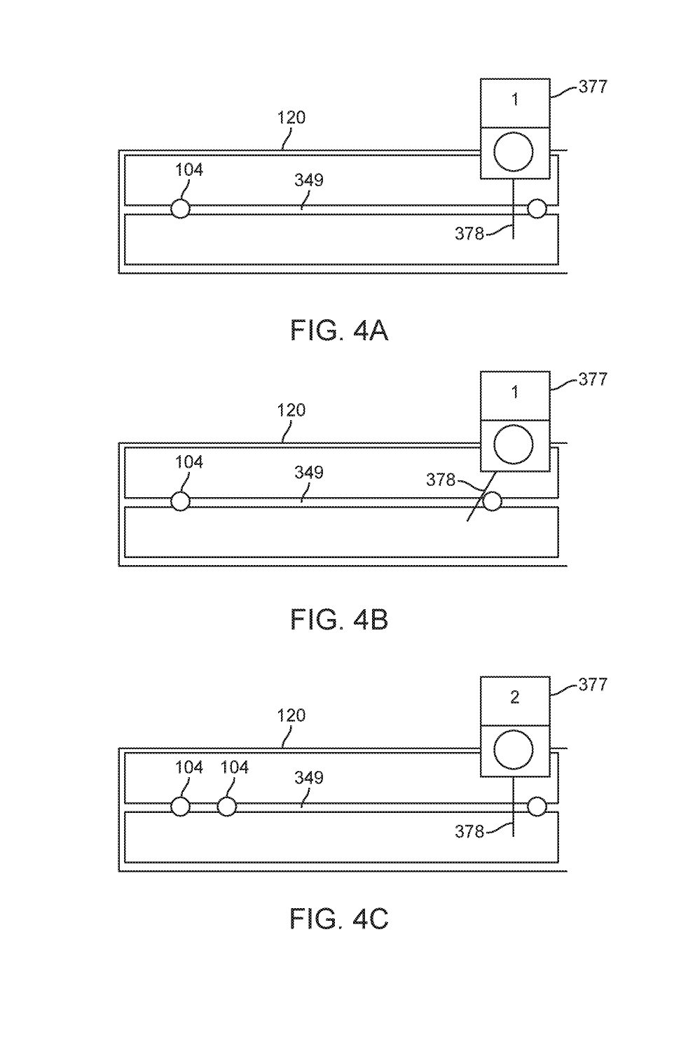

With reference to FIGS. 4A-4C, in an embodiment, mechanical counter devices can be used with the needle dispensing unit 110 and/or the needle receptacle 120 to facilitate needle counting. Additional circuitry such as a processor and wireless communication circuitry can be coupled to the counter to track usage of the needles and to transmit the unique identifiers and time stamp data as described herein. In the illustrated embodiment, an arm can be actuated to cause a numerical indicator to advance the number displayed. In FIG. 4A, a single needle 104 has been placed in the needle receptacle 120 and the visual display 377 shows “1”. With reference to FIG. 4B, a second needle 104 can slide through the needle slot 349 and contact the arm 378 which rotates about an axis and actuates the visual display 377 to advance the displayed number. With reference to FIG. 4C, after the second needle 104 passes the arm 378, the display 377 has changed to “2” and the arm 378 has reset to its normal position detect the next needle 104. A similar counting system may be employed with the needle dispensing unit, in which a mechanical arm is actuated by each dispensed needle, causing a numerical indicator to advance, thereby counting each needle dispensed.

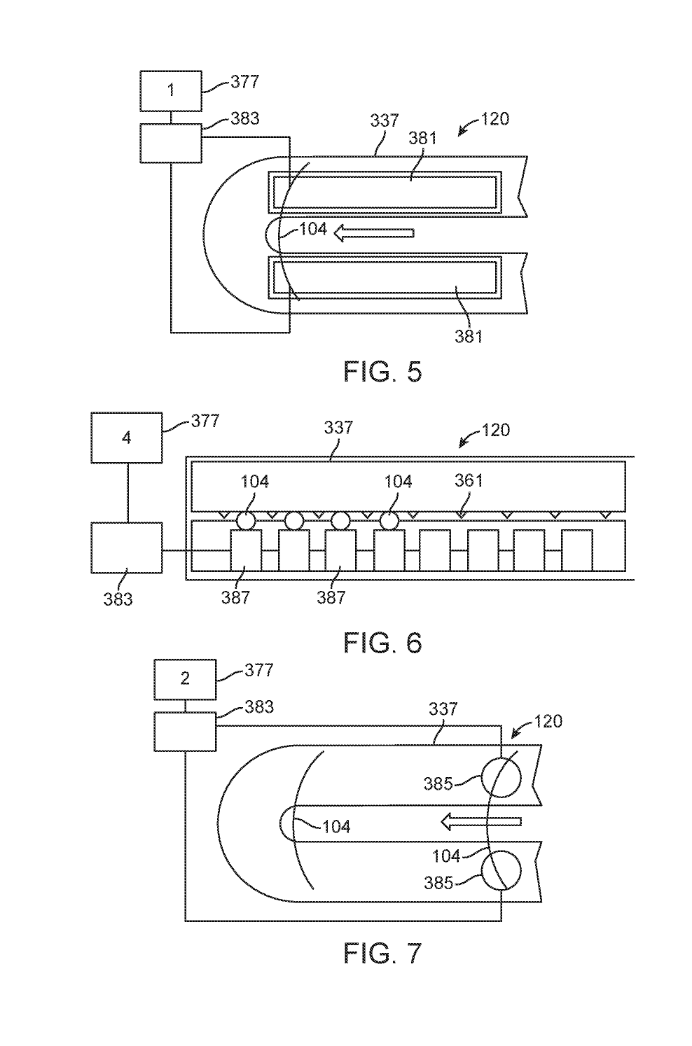

In an embodiment with reference to FIG. 5, an optical counter mechanism can be used with the needle receptacle 120 to indicate the number of stored needles 104. An optical scanner(s) 381 can be used to detect the number of needles 104 that are stored in the secure zone 337 of the needle receptacle 120. The scanner 381 may also be designed to operate in other areas of the radio frequency spectrum such as infrared, UV, radar etc. for the counting function. In another embodiment, a reflective scanner can be used to quantify amount of metal from strength of reflected or transmitted optical signal. In an embodiment an infrared image can detect needles in the needle receptacle 120 with better accuracy than visual counting from a standard optical image of the needle receptacle 120. The plastics and foam components of the needle receptacle 120 can transmit infrared energy whereas the metal needles 104 can reflect the infrared energy. The optical scanner 381 can transmit scanned needle information to a processor 383 that can convert the scanned signal into a number representing the number of needles 104 in the secure zone 337 of the needle receptacle 120. The processor 383 can be coupled to a visual display 377 that can be controlled to display the number of detected needles in the secure zone 337 of the needle receptacle 120. Additional circuitry such as wireless communication circuitry can be coupled to the processor to track usage of the needles and to transmit the unique identifiers and time stamp data as described herein.

With reference to FIG. 6, in an embodiment the system can detect the number of needles in the secure zone 337 of the needle receptacle 120 based upon pressure measurements detected by transducers 387. In the illustrated embodiment, the needle receptacle 120 transducers can detect compressions in the compressive member 347 caused by the needles 104. The transducers 387 can be positioned along the length of the secure zone 337 and the protrusions 361 can create individual needle storage areas. By measuring the increased pressure in each of the needle storage areas, the number of needles 104 in the secure zone 337 can be determined. The transducers 387 can be coupled to a processor 383 which can determine the number of used needles 104 in the secure zone 337 based upon the transducer 387 signals and the processor 383 can transmit a needle count number signal to the visual display 377 which can display the needle count number. In different embodiments, different types of transducers 387 can be used to detect the needle pressure. For example, the transducers 387 can be can be piezoelectric devices that can also be used in which pressure applied to compressive member 347 and records the presence of each needle 104. Alternatively, the transducers 387 can include a series of strain gages that may be utilized to sense the presence of needles 104 in the secure zone 337 or any other suitable pressure detecting mechanisms. A similar tracking system may be employed with the needle dispensing unit, to track needles in the suture pack and as they are dispensed. In such a system, transducers 387 coupled to a processor 383 track each of a plurality of needles from a suture pack by detecting the presence or absence of needles as described above. By combining these two systems, needles can be tracked continuously both before being dispensed and after being secured. Additional circuitry such as wireless circuitry can be coupled to the processor to track usage of the needles and to transmit the unique identifiers and time stamp data as described herein.

With reference to FIG. 7, an optical sensor such as camera(s) 385 can be used to detect the number of needles 104 that move into the secure zone 337 of the needle receptacle 120, as well as to detect needles as they are dispensed from the needle dispensing unit 110. The cameras can be coupled to a processor 383 that receives needle count signals as each needle 104 passes over the optical sensor such as camera(s) 385. The processor can count and store the needle count signals and output a needle count signal to the visual display 377 which can display the number of detected needles 104 in the secure zone 337 of the needle receptacle 120. In different embodiments, different types of cameras 385 can be used. For example, the needles 104 can be more visible to an infrared sensor than a visual wavelength optical camera. Thus, an infrared camera 385 may more accurately detect the movement of needles 104 into the secure zone 337. Similarly, one or more cameras may be employed to detect the number of needles 104 dispensed by the needle dispensing unit 110. Additional circuitry such as wireless circuitry can be coupled to the processor to track usage of the needles and to transmit the unique identifiers and time stamp data as described herein.

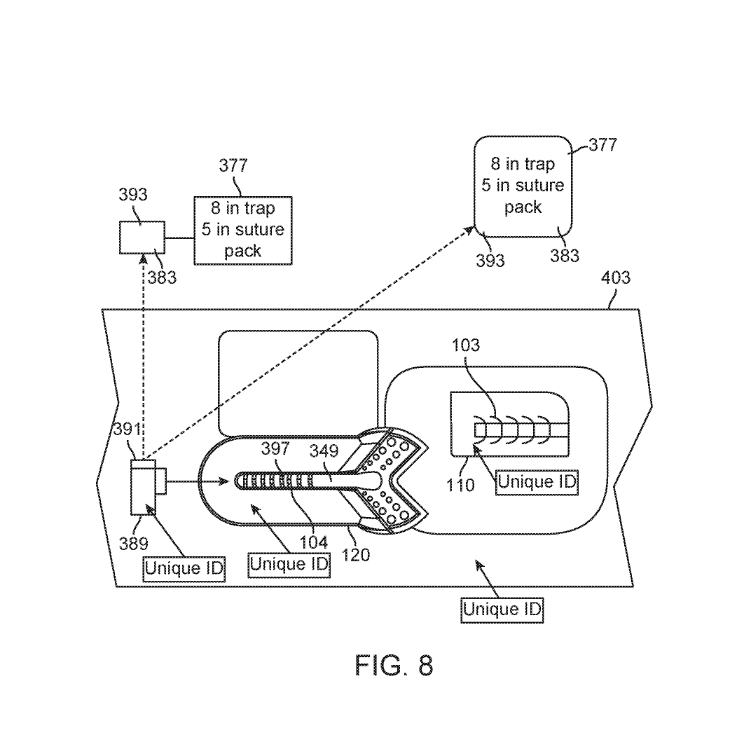

With reference to FIG. 8, in other embodiments, the needle receptacle 120 and/or needle dispensing unit 110 can be used with other components to perform needle counting. In the illustrated example, the needle receptacle 120 can be mounted on a barrier 403 as described herein that can be placed on a forearm of a surgeon. A needle sensor 389 can detect needle count signals and the needle count signals can be transmitted by a transmitter 391 to a receiver(s) 393 which can be coupled to a processor(s) 383 which can output needle count information to an output device 377 which can indicate the number of needles in the needle receptacle 120. In the illustrated embodiment, the needle sensor 389 can be a small camera with an integrated radio frequency (RF) transmitter 391 which transmits image and/or video RF signals to receivers 393. Processor(s) 383 coupled to the receivers 393 can output image and/or video signals to visual displays 377 which can display the needle driver slot 349 to allow the needles 104 to be visually counted remotely. The needle sensor 389 and transmitter 391 can be within the near surgical field. In contrast, the receivers 393, processors 383 and visual displays 377 can be well outside the near surgical field. One or more of the needle receptacle 120 such as a needle trap, the needle dispensing unit 110 such as a suture pack, the needle sensor 389, the barrier 403, or the sensor 389 may comprise a unique identifier (ID) as described herein. The processor 383 may comprise instructions to timestamp the received data. The barrier may have the processor and a power supply mounted thereon, for example.

The camera can face the needle receptacle 120 and needle dispensing unit 110. The images of the needle receptacle 120 and needle dispensing unit 110 can be transmitted to the visual display(s) 377 which can be visible to another person. For example, the remote visual display(s) 377 can be a video display mounted on an operating room wall. As discussed, a portion of each of the needles 104 may be visible from the upper surface of the needle trap 120 through at least the needle driver slot 349. Thus, a displayed image of the needle receptacle 120 and needle dispensing unit 110 on the surgeon's forearm can show the number of used needles 104 in the needle receptacle 120, as well as the number of new suture needles 103 dispensed from a suture pack by the needle dispensing unit 110. A surgical assistant can view the display 377 and see the needle dispensing unit 110 and the needle receptacle 120 with the secured needles 104 to track in real time. The surgical assistant can then provide additional suture packs for the needle dispensing unit 110 if additional needles 103 are required and provide new empty needle receptacle 120 as the barrier mounted needle receptacle 120 become full of used needles 104 and needs to be replaced. Also, if a needle 104 is lost the error can immediately be detected by someone monitoring the surgical procedures or by the processor which can detect the sequential removal of new needles 103 from a suture pack in the needle dispensing unit 110 and the delivery of the used needles 104 to the needle receptacle 120. Although an exemplary set of system components has been described, in other embodiments, the needle count components can include but are not limited to: dedicated receivers, electronic watches, smartphones, tables, computers, headsets, earpieces, displays, or any other suitable device for the purpose of tracking the needles.

As discussed, mid-bodies of needles 104 may be visible through the needle driver slot 349 in the needle receptacle 120. In an embodiment, the processor 383 can run a software program that can interpret and time stamp the visual display signals from the needle sensor 389 (camera) and determine the number of needles 104 in the needle receptacle 120 as well as the needles 103 in the suture pack 110. The processor 383 can then output this needle count number on the visual display 377 which can help with the needle counting process. In other embodiments, the needles 104 can include markings 397 or transmitters that can help track the needles 104. In an embodiment, the markings may comprise visual codes such as bar codes, quick response (QR) codes, color codes, numeric markings or any other markings which can provide at least some identification information about the needles 104. The markings can be placed on the middle body portion of the needles 104. When the needles 104 are placed in the needle receptacle 120, the markings can be visually detected through the needle driver slot 349 in the needle receptacle 120 by an optical sensor such as a scanner or a camera. In an embodiment, an optical needle sensor 389 can detect the markings and the processor 383 can interpret the markings and determine the identifications of the needles 104 based upon the markings. This identification information can then be used for needle tracking and needle reconciliation. The identification information can also be output to the visual display 377.

In other embodiments, other mechanisms can be used for needle tracking. For example, in an embodiment the needles 104 can include embedded electronic components such as a radio frequency transmitter such as a radio frequency identification tag (RFID) which can transmit an RF identification signal in response to exposure to an interrogating radio wave. In an embodiment with reference to FIG. 8, the needle sensor 389 can include an interrogating radio wave transmitter and an RF receiver. When exposed to the interrogating RF waves, the RFID tags on the needles 104 can emit RFID signals that can be detected by the RF receiver. The RFID information can be transmitted to the processor 383 which can then identify each needle in the needle receptacle 120.

In other embodiments, the needle dispensing unit 110 can also have integrated tracking mechanisms. For example, the suture packs or needle dispensing unit can include an active electronic sensor that can be activated when needles are dispensed. This active signal can be transmitted to a processor off the surgical field that can monitor the use of the needle dispensing unit and know which needles must be reconciled after being dispensed from the suture pack by the needle dispensing unit. In an embodiment, these active signals can be transmitted wirelessly from a needle dispensing unit or suture pack sensor to a remote receiver. These active signals can be processed by a processor as described above. This feature can allow the needles to be tracked from the suture pack to the needle receptacle in a closed loop manner to further ensure that all needles are accounted for.

In another embodiment, the tracking of the needles can be done more locally on the barrier which can be mounted on the forearm of the surgeon. In this embodiment, a processor can be mounted on the barrier and the processor can keep track of the locations of all needles throughout the surgical procedure. An active signal can identify a suture pack that is being opened and the identities of all of the needles in the newly opened suture pack. The system can identify the movement of each of the needles as they are dispensed by the needle dispensing unit from the suture pack through a patient and into the needle receptacle. If a needle is lost the processor that can output an error signal to an output device such as a visual display or audio output device can immediately detect the error. If possible, the surgical procedure can be temporarily stopped until the lost needle is found. The described needle tracking can also provide useful needle tracking information that can be stored in a data center and the number of needles in the near surgical field can be automatically reconciled in real time. As needles are secured in the needle receptacle, the system can broadcast correlation information for needle reconciliation.

In further embodiments, the tracking systems described herein may be used interchangeably for tracking of needles as they are dispensed from needle dispensing unit 110 and secured by needle receptacle 120. For example, dispensed needles might be tracked by piezoelectric pressure measurements while returned needles are tracked by actuated levers, or vice versa. It will be understood that the various tracking mechanisms described herein may be freely and interchangeably chosen for tracking needles in either direction. Additionally, multiple such tracking mechanisms may be used redundantly; for example, needles may be tracked by both RFID and optically, by both the needle dispensing unit and the needle receptacle. Each of the tracking mechanisms may comprise an associated unique identifier, and may be configured to timestamp the data.

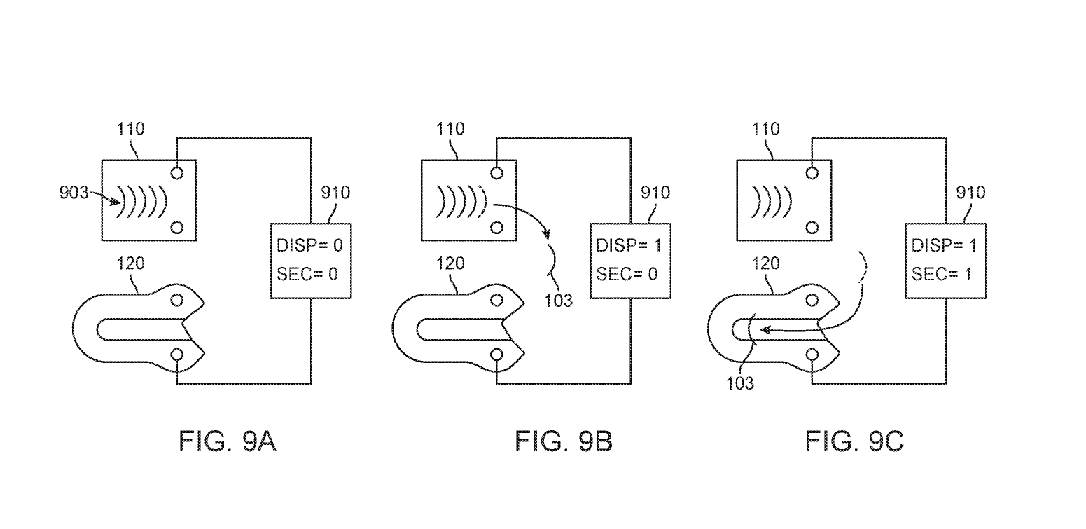

FIGS. 9A-C illustrate an overview of needle tracking, in which the dispensing and securing of needles is reconciled. FIG. 9A shows a needle dispensing unit 110 and a needle receptacle 120 each connected to a circuit containing a counter 910. The counter tracks each needle dispensed from the needle dispensing unit 110 and each needle received by the needle receptacle 120 using signals transmitted from each. FIG. 9A shows this system in its initial state, in which no needles have been dispensed or secured. Accordingly, the counter shows 0 needles dispensed and 0 needles secured. A plurality of needles 903 are illustrated in a suture pack in the needle dispensing unit 110. The fact that the number of needles dispensed is equal to the number of needles secured indicates that all needles have been accounted for, and that it is safe to dispense a needle from needle dispensing unit 110.

FIG. 9B illustrates a second system state, in which a needle 103 has been dispensed by needle dispensing unit 110 for use in a surgical procedure. As the needle 103 is being dispensed by the needle dispensing unit 110, its removal is detected, for example, using one of the methods for needle tracking disclosed above. In response, a signal is sent to the counter 910, causing the counter 910 to increment its count of needles dispensed to 1. Because the number of needles dispensed is not equal to the number of needles secured—in particular, it is greater—this indicates that not all needles are accounted for. For this reason, in systems employing an optional “lock-up” mechanism, the needle dispensing unit may be inhibited from dispensing needles until the counter concludes that the number of dispensed and secured needles matches. This inhibition may be accomplished, for example, by a signal between counter 910 and needle dispensing unit 110 indicating whether the system has or has not secured all needles.

FIG. 9C illustrates a third system state, in which the needle 103 has been placed into the needle receptacle 120. As the needle 103 is secured in the needle receptacle 120, it is detected, for example, using one of the methods for needle tracking disclosed above. In response, a signal is sent to the counter 910, causing the counter 910 to increment its count of needles secured to 1. Because the number of needles secured is now equal to the number of needles dispensed, this indicates that all needles are accounted for. Accordingly, in systems employing an optional “lock-up” mechanism, the needle dispensing unit may be released, allowing it to dispense needles again. This release may be accomplished, for example, by a signal between counter 910 and needle dispensing unit 110, indicating that all needles are secured and that further needles may be dispensed. Alternatively, if a signal is used to inhibit needle dispensing when number dispensed and number secured are unequal, that signal may be terminated, allowing needle dispensing unit 110 to continue dispensing needles.

The counter 910 may conveniently comprise a processor with associated memory containing instructions that, when executed, cause the counter 910 to respond to signals from the needle dispensing unit 110 and needle receptacle 120 as described above, including, for systems using a “lock-up” feature, sending appropriate control signals to the needle dispensing unit. These signals may be sent using electrical circuits, as illustrated. Alternatively, other methods of signal transmission may be employed, such as wireless communication.

In systems employing a “lock-up” feature, there should only be one or zero needles in use at any given time. For this reason, it may be desirable to employ simpler control circuitry with two operating states: “open” and “locked.” An “open” state corresponds to zero needles in use, and indicates that a new needle may be dispensed. When a needle is dispensed, the state is switched to “locked.” A “locked” state indicates that a needle is in use, and that the needle dispensing unit is to be inhibited from dispensing any more. When a needle is returned to the needle receptacle, the state toggles back to “open.” Although a processor can be used in this manner, the small number of states needed when operating in this manner allows this behavior to be controlled by simple electronic circuitry; for example, a gate array or similar printed circuit chips may be employed. Systems employing a “lock-up” may also include a reset mechanism, such as a reset button, to allow the system to be unlocked without inserting a needle. The activation of this mechanism switches the system to the “open” state regardless of its current state.

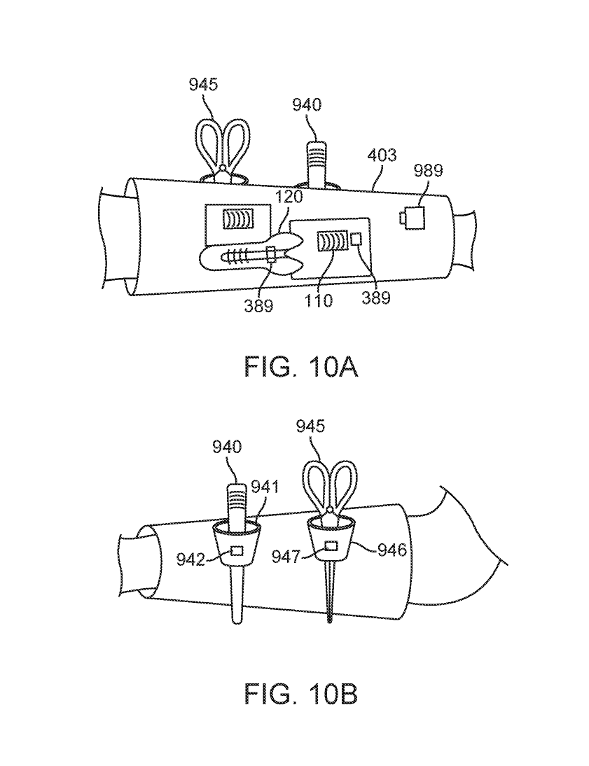

FIGS. 10A and 10B show volar and dorsal views, respectively, of a forearm-mounted barrier 403. FIG. 10A shows a volar view. On the volar side, the barrier comprises a needle dispensing unit 110 such as a suture pack and a needle receptacle 120, each with corresponding sensors 389 to track dispensation and securing of needles. Additional sensors 989 are illustrated, and may comprise optical, radio frequency, or other tracking mechanisms to track needle usage, using methods as described herein. Each of the sensors may comprise a unique identifier as described herein. Each of the barrier 403, needle dispense unit, and needle receptacle may comprise a unique identifier as described herein. Each of these units is disposed on a forearm-mounted barrier 403, which serves both to hold the various components in a convenient location as well as to provide a barrier to protect the arm of the surgeon from puncture injuries. Further visible are tools 940 and 945, secured on the dorsal side of the barrier, and shown in more detail in FIG. 10B. Circuitry such as wired or wireless communication circuitry can be provided on the barrier to transmit information from the sensors, and a power supply can be supported with the barrier to power the circuitry on the barrier. Alternatively, the barrier may comprise connectors that connect to a power supply.

FIG. 10B shows a dorsal view of forearm-mounted barrier 403. The dorsal side comprises tool receptacles 941 and 946 for holding tools 940 and 945, respectively, for use in surgical procedures. The receptacles each comprise sensors 942 and 947 for tracking tool usage. The sensors are configured to track when each tool is removed from or inserted into its respective receptacle. This tracking may be done in various ways, depending on the type of sensor chosen. For example, the sensor may be a lever that is actuated by a tool inserted into its receptacle. The state of the tool is then indicated by the position of the lever. Another option is a sensor such as an optical, laser, or infrared sensor, by which the presence of an object within the receptacle may be determined. Each sensor may comprise circuitry to transmit data, such as wireless communication circuitry, for example. Further sensor possibilities include RFID sensors, cameras, barcode or QR code sensors, etc. These types sensors have the added benefit that the identity of each tool may be tracked, for example, by assigning each tool a unique RFID chip, color, shape, code, etc., as appropriate. Each sensor may be configured to transmit data representative of its measurements to a processor, so that tool usage may be monitored and recorded in real time. By tracking continuously the usage of tools, as well as needle usage, throughout a surgical procedure, an accurate and detailed picture may be constructed of the surgical workflow throughout the procedure.

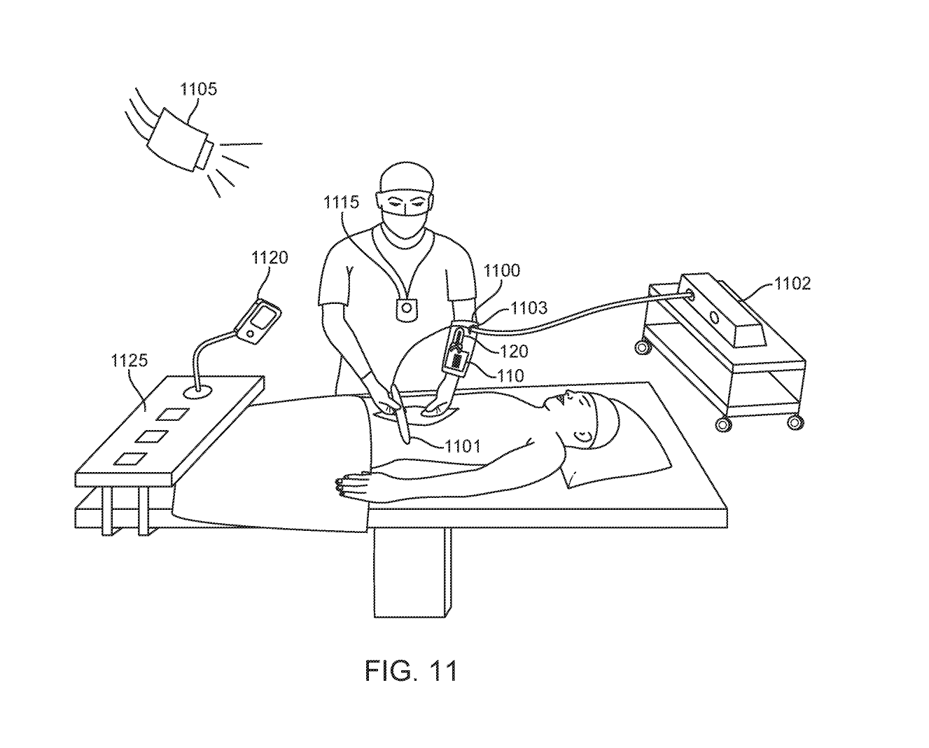

FIG. 11 illustrates an overview of the data tracking enabled by the use of the systems and devices disclosed herein. The surgeon of FIG. 11 is shown holding an electrocautery pen 1101 in his dominant right hand for use in a surgical procedure. The surgeon wears a needle tracking system 1100 on his non-dominant left arm. The needle tracking system 1100 comprises a needle receptacle 120 and needle dispensing unit 110 on a forearm-mounted barrier, as disclosed above. The tracking system further comprises plugs 1103 for connection to the electrocautery pen 1101, as well as to an electrocautery machine 1102 which provides electrical power through a connected cable for use in an electrocautery procedure. As the surgeon uses the electrocautery pen 1101, its use is monitored by the tracking system. This monitoring may be performed, for example, by the tracking system 1100, through which electrical current may be directed using the plugs 1103. The flow of current and voltage drop over the electrical circuit comprising the electrocautery pen may be continuously monitored, allowing the continuous determination of the electrical power being used to heat the patient's tissue by electrocautery pen 1101. This recorded data may be stored in memory and/or transmitted to a remote server for use in later analysis of the surgical procedure. In some embodiments, the electrical power usage may be monitored directly by the electrocautery machine 1102. In this case, it may not be necessary to run electrical power through the tracking system 1101, but instead, a wire may lead directly from the electrocautery machine to the electrocautery pen. Each recording device may transmit its recorded data to a server for analysis. This transmission may be accomplished using a communications network, including wireless connections such as Bluetooth, cellular, or other wireless communications. The transmitted data may be collected and stored, using a database, for example, to enable analysis of workflow and energy usage throughout a surgical procedure.

To enable further accurate monitoring of surgical workflow, recording devices may be provided throughout the surgical environment of the operating room. Cameras 1105 disposed about the operating room may provide continuous video recording of the surgical procedure. Such cameras may, for example, be mounted on operating room walls, or on movable stands, allowing the cameras to be disposed at locations to conveniently capture video of the surgical procedure. The cameras may also incorporate lighting to illuminate their fields of view. Additional recording devices may be located in the operating room, such as recording device 1115 worn by the surgeon to record audio and/or video of the procedure. Mobile devices such as tablets or smartphones may be conveniently used as such recording devices, and a sterile case may be provided to allow their safe use in an operating room. A mobile device 1120 is also illustrated disposed in a sterile cover connected to a flexible stand 1125 such as a Mayo stand, allowing it to be maneuvered to obtain clear images of the surgical procedure. The sterile cover may comprise one or more of a case or container, such as a sterile bag that receives the mobile devices. The flexible stand may comprise a USB or other connection, to provide power and/or data transmission capability to a connected mobile device. Audio may also be recorded by the mobile device 1120 or by other audio recording devices. Audio recording may in some cases be continuous throughout the procedure, or alternatively performed only as needed, for example, using voice commands, buttons, etc. to toggle recording on and off. Similarly, video recording may be performed continuously, while also allowing for surgeons and/or surgical staff to indicate particular moments or time periods of interest. For example, a voice command or pressed button or switch may be used to cause one or more snapshots to be recorded by one or more of the recording devices. Each recording device may be connected to a communications network to allow transmission of its recorded data to a central server for analysis and storage.

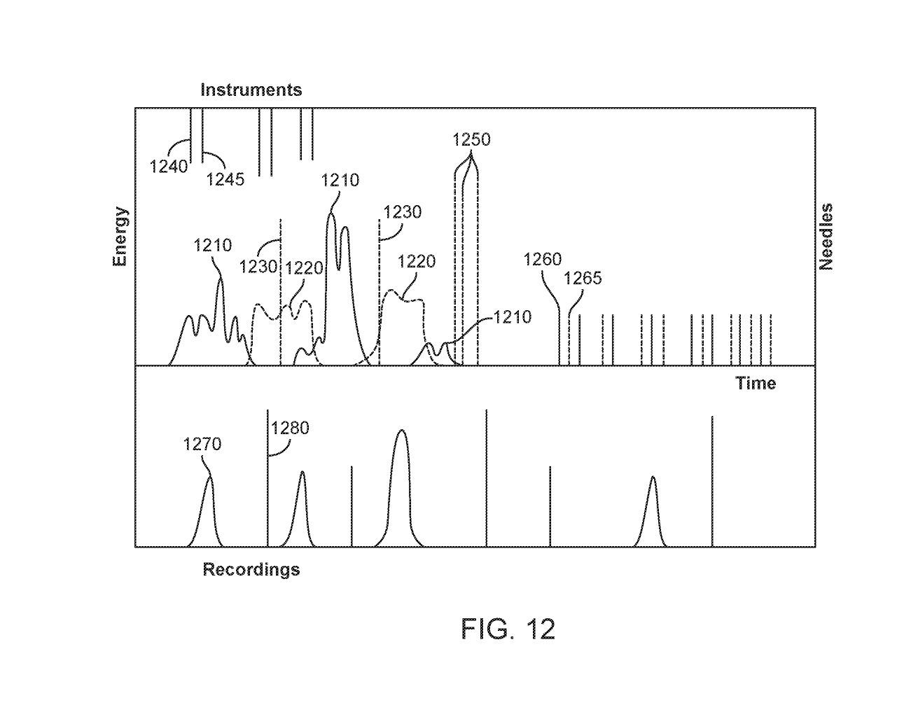

The recorded data as described herein may be used to provide a comprehensive understanding of surgical procedures. For example, FIG. 12 shows a graphical representation of data that may be recorded during a surgical procedure. The data can be recorded with appropriate time stamps that are registered to provide the output graph with a common time base. The data recorded are illustrated as a function of time, with the upper panel representing both the use of surgical tools and the application of energy in various forms to the patient during the surgical procedure and the lower panel representing recordings of video and audio. In the upper panel, curves showing amount of energy applied per unit time is shown in surgical procedures such as electrocautery 1210, fluoroscopy 1220, and X-ray imaging 1230 is shown with graphical representation of amount of applied power for each as a function of time. Each curve is generated by a monitoring process such as that described above for electrocautery, and the recorded data are collected by a central server connected to devices such a electrocautery machines, fluoroscopes, and X-ray machines, each of which may monitor its own amount of power applied to the patient. Each curve may be integrated to determine a quantity proportional to its total respective energy applied by each source. The recording and display of each curve is individually optional, and additional curves may be recorded for further devices that may apply energy to the patient, such as surgical lasers, ultrasound probes, electromagnetic radiation or particle pulses, etc. In the case of short pulses such as X-rays, which are typically recorded briefly and are shown as sharp peaks in FIG. 12, a single value may in some cases be recorded for each peak, representing its respective total energy, such as X-ray energy, applied. Each type of energy signal is recorded separately, but in some cases may be combined; for example, the total amount of X-ray and fluoroscope usage may be integrated together using appropriate weighting parameters to determine a total exposure to ionizing radiation from these sources. In addition to energy measurements, other metrics may be recorded during the surgical procedure. For example, flow of anesthesia, transfused blood, antibiotics, anticoagulants, and other intravenous fluids into the patient may be recorded by flowmeters. Gas input such as oxygen may be similarly recorded. Patient vital indicators may also be monitored and recorded, such as blood oxygenation, pulse, electroencephalogram (EEG), electrocardiogram (EKG), respiration, body temperature, and blood pressure. Additionally shown in the top panel is tool usage, as monitored by the tracking system described herein. Each removal 1240 and replacement 1245 of each tool from tool receptacles may be recorded, with different tools represented by lines of different length. Additionally, the implanting of objects such as catheters, pacemakers, artificial or transplanted organs, artificial joints, surgical pins, rods, screws, or plates, etc. may be monitored and recorded, as shown by implant lines 1250. In some cases, the insertion of implants may be determined from video or X-ray images. Alternatively or additionally, the surgeon or surgical staff may indicate the times at which each implant is inserted, for example, by speech, actuation of a lever, pressing of a button, etc. At the end of the procedure, the surgeon closes using a plurality of needles to suture the patient. Each time a needle is dispensed 1260 or secured 1265, the system records that fact as well as the time it happens, allowing a graphical representation of exactly how much time is spent between each suture added to the patient. This can help in understanding surgeon workflow and give insight into how to optimize surgical procedures and cut down on wasted time. Data from different surgeons may be compared, as well as data from the same surgeon performing either similar or dissimilar procedures, allowing trends to be detected; for example, surgical efficiency may vary depending on time of day, day of the week, time between operations, total time spent so far in a shift, etc. Correlations may also be found between, for example, patient vital signs and particular steps of surgical procedures; for example, the use of certain tools may correlate with changes in blood pressure or oxygenation, either because their use causes the changes, or because they are used in response to such changes.

The lower panel of FIG. 12 shows a parallel recording of video and audio data that may be produced during a surgical procedure, illustrated on the same time axis as the upper panel. The audio recordings 1270 are shown, indicating total audio volume for each time period that recording is turned on. The audio recordings may be played back at a later time, for example, to review notes made by the surgeon and/or surgical staff during the procedure. Video may also be viewed of the procedure, along with or without the associated audio recordings. Images recorded by the surgeon or surgical staff are also shown by image markers 1280; these images may for example be snapshots of important moments in the surgical procedure. In some cases, the audio recording may include description of the images before, after, or during the time that the images are recorded. All of the data shown here may be combined by appropriate computing systems, and recorded, for example, in a database which may be accessed after the procedure for review and analysis. The data may be made available on a mobile device, such as a tablet or smartphone. The data may even be made available in real time during a surgical procedure; for example, on mobile device 1120. In order to allow the combination of all data inputs, each input device may be synchronized to a common time base, so that, as shown in FIG. 12, each marker line and curve may be associated with a particular time or period of time.

The graphical representation can be shown on a display of a user device. The graphical display can be interactive and allow the user to obtain additional detail on each of the structures of the report. The structure of the report may comprise one or more items shown on graphical representation the display, such as an image. The user may touch on one of the items to view additional detail, for example by touching an appropriate item on a touch screen display.

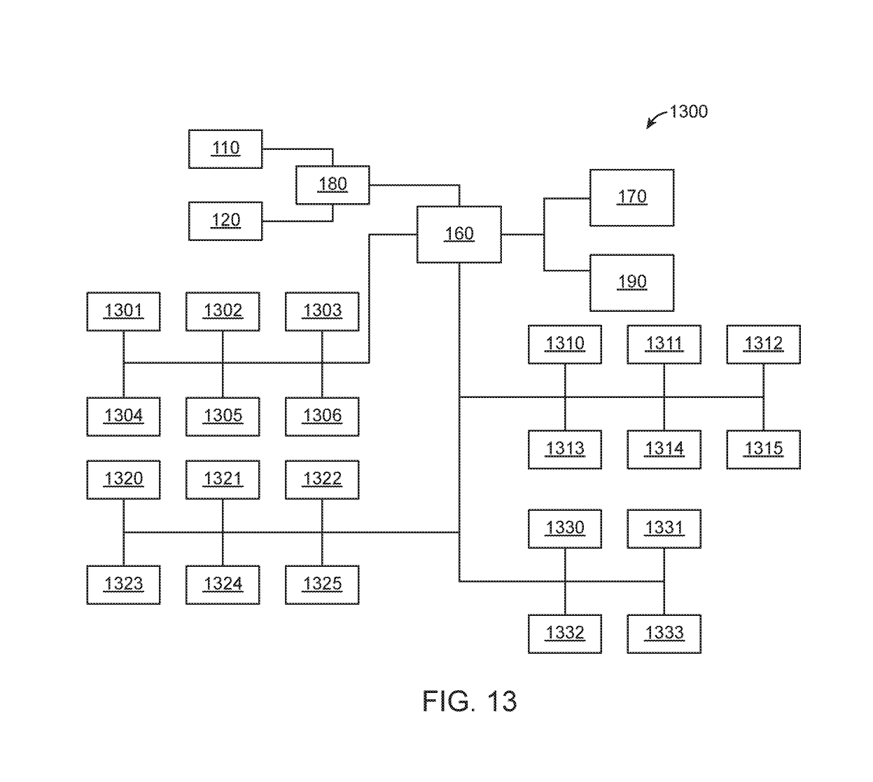

FIG. 13 illustrates a schematic diagram of an exemplary system 1300 for surgical workflow monitoring, in accordance with embodiments. As illustrated in FIGS. 2A and 2B above, the dispensing and securing of needles by needle dispensing unit 110 and needle receptacle 120, respectively, as well as the usage of tools from tool receptacles are tracked by one or more sensors 180, which communicate the tracking information to a processor 160. Also connected to the processor are further devices and modules, each providing data to be processed. For example, electrocautery device 1301 may measure quantities such as voltage and current so as to determine electrical power usage, and communicate these values to the processor 160. X-ray device 1302 records and transmits the timing of X-ray images, as well as an associated intensity and/or pulse duration. Fluoroscope 1303 records and transmits data representative of radiation intensity as a function of time. Ultrasonic probe 1304 records and transmits data representative of ultrasonic pulse duration and intensity. Surgical laser 1305 records and transmits data representative of laser pulse duration and intensity. Radiation therapy device 1306 records and transmits data representative of the amount of radiation applied as a function of time. Further devices may be used to assess quantities of fluids and gases to the patient as a function of time. Anesthesia administering device 1310 measures the amount of each anesthetic administered to the patient, for example, by recording and transmitting flow rates for each anesthetic as a function of time. Blood transfusion device 1311 measures the amount of blood administered to the patient, for example, by recording and transmitting flow rates from a source of blood for transfusion. Further devices that may be used include devices to administer antibiotics 1312, anticoagulants 1313, and other intravenous fluid dispensers 1314. Each such device monitors its respective flow as a function of time and transmits corresponding data to the processor 160. Gas flow to the patient may be monitored and transmitted to the processor 160, for example by oxygen dispenser 1315. Generally, gas and liquid flow may be measured by devices such as mechanical or electrical flowmeters or by monitoring total remaining fluid as a function of time. Gas flow may also be measured in other ways, such as monitoring gas pressure across a regulator to estimate flow.

Patient vital signs may be monitored as appropriate during a surgical procedure. Monitoring devices include blood pressure meter 1320, pulse oximeter 1321, EEG device 1322, EKG device 1323, respiration monitor 1324, and thermometer 1325. Each device records continuous data over time and transmits its readings to the processor 160. Further recording devices, such as audio recorders 1330, video cameras 1331, digital cameras 1332, and mobile devices 1333 record audio, video, still images, or a combination thereof, and transmit corresponding data to the processor 160. The processor 160 receives data from each connected device and records corresponding information in memory, such as database 190. The processor 160 also produces graphical representations of the recorded data, such as those shown in FIG. 12, for display in display device 170. Display device 170 may, for example, be a mobile device in the operating room, such as mobile device 1220, allowing real time display of relevant data during a surgical procedure.

As disclosed in further detail herein, each connection to the processor may independently be wired or wireless, as needed to ensure reliability and accuracy of data transmission. The data may also be transmitted in a secure format, for example, using data encryption, to protect confidentiality. Time is also continuously tracked for each device connected to the processor, either locally using a synchronized clock, or globally by a clock associated with the processor 160. For devices using the processor clock, timing information may be based on the time data are received by the processor. For locally timed devices, timestamps or other timing data may be transmitted along with signal data.

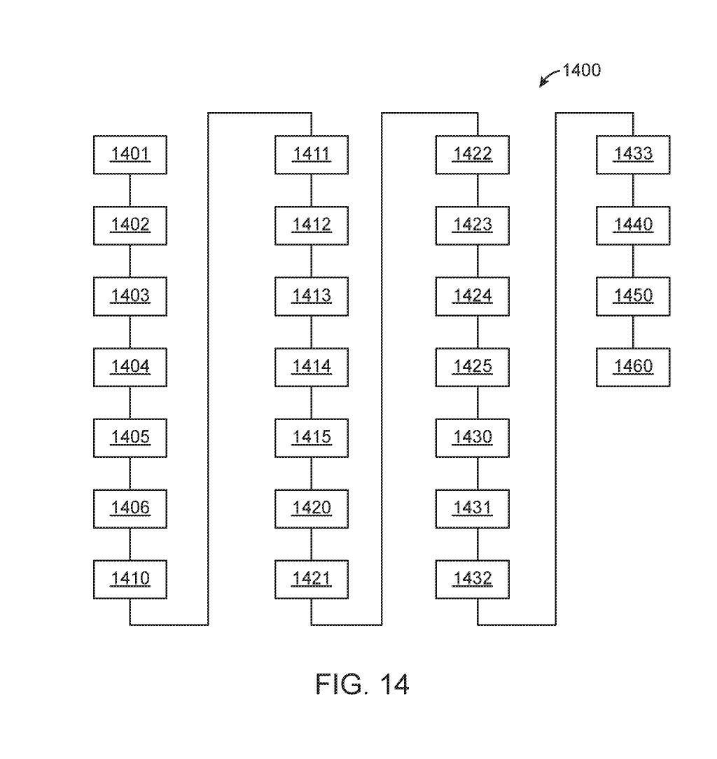

FIG. 14 illustrates a surgical workflow monitoring method 1400, according to embodiments. The steps of method 1400 may be performed by a processor 160 connected to each of a plurality of surgical devices to monitor device usage throughout a surgical procedure. Each of the steps 1401 through 1460 may be performed repeatedly, as a loop, to accumulate and display further surgical data over the course of a procedure. The order of steps may be permuted as needed, and individual steps may be omitted when not needed.

In step 1401, the processor 160 receives data from an electrocautery machine 1301 indicating the quantity of power, if any, applied during the period since the last update.

In step 1402, the processor 160 receives data from an X-ray machine 1302 indicating whether an X-ray was taken and, if so, how much radiation was applied to the patient.

In step 1403, the processor 160 receives data from a fluoroscope 1303 indicating the quantity of fluoroscope radiation, if any, applied during the period since the last update.

In step 1404, the processor 160 receives data from an ultrasonic probe 1304 indicating the quantity of acoustic energy, if any, applied during the period since the last update.

In step 1405, the processor 160 receives data from a surgical laser 1305 indicating the quantity of laser power, if any, applied during the period since the last update.

In step 1406, the processor 160 receives data from a radiation therapy device 1306 indicating the quantity of radiation, if any, applied during the period since the last update.

In step 1410, the processor 160 receives data from anesthesia machine 1310 indicating the quantity and types of anesthesia administered to the patient during the period since the last update.

In step 1411, the processor 160 receives data from blood transfusion device 1311 indicating the quantity of blood, if any, administered to the patient during the period since the last update.

In step 1412, the processor 160 receives data from antibiotic administration device 1312 indicating the quantity and types of antibiotics, if any, administered to the patient during the period since the last update.

In step 1413, the processor 160 receives data from anticoagulant administration device 1313 indicating the quantity and type of anticoagulants, if any, administered to the patient during the period since the last update.

In step 1414, the processor 160 receives data from intravenous fluid dispenser 1314 indicating the quantity and type of intravenous fluid, if any, administered to the patient during the period since the last update.

In step 1415, the processor 160 receives data from gas administration device 1315 indicating the quantity and type of gas, such as oxygen, administered to the patient during the period since the last update.

In step 1420, the processor 160 receives data from blood pressure meter 1320 indicating the blood pressure of the patient during the period since the last update.

In step 1421, the processor 160 receives data from pulse oximeter 1321 indicating the blood oxygenation and/or pulse rate of the patient during the period since the last update.

In step 1422, the processor 160 receives data from EEG device 1322 indicating the EEG of the patient during the period since the last update.

In step 1423, the processor 160 receives data from EKG device 1323 indicating the EKG of the patient during the period since the last update.

In step 1424, the processor 160 receives data from respiration monitor 1324 indicating the breathing rate of the patient during the period since the last update.

In step 1425, the processor 160 receives data from thermometer 1325 indicating the body temperature of the patient during the period since the last update.

In step 1430, the processor 160 receives audio recording data from audio recording device 1330.

In step 1431, the processor 160 receives video recording data from video recording device 1331.

In step 1432, the processor 160 receives image data from digital camera 1332.

In step 1433, the processor 160 receives data from mobile device 1333. This data may comprise video, audio, and/or still image data. The data may further comprise user instructions sent to the processor; for example, requesting the processor to provide a graph of one or more curves for display.

In step 1440, the processor receives data from sensors on a forearm-mounted barrier, indicating the status of the count of dispensed and secured needles, including whether a needle has been dispensed or secured since the last update. Tool usage data is likewise transmitted, indicating whether each tool has been removed from or returned to its receptacle. Identifying information sufficient to determine the identity of the barrier, as well as that of the surgeon or surgical staff member using the barrier may also be received. The identifying information may be information that was input into the barrier, or into a mobile device such as mobile device 1120, 1115, and/or 1333. Alternatively, the identifying data may be received from an external network source, such as an operating room scheduling database. In some cases, the identity of the user and/or the barrier may be determined by analysis of video recording the surgical procedure. The receipt of this data allows the association of a given set of surgical workflow data with both a particular barrier, as well as the individual performing the surgical procedure.

In step 1450, the processor processes the data it has received from each of the devices in steps 1401 to 1440. For each measured quantity, the processor updates a corresponding database entry to record the data received. Each entry indicates the updated status of its respective measurement, as well as associating a time with that status.

The data input to the processor can be timestamped for the processor to provide a common time base.

In step 1460, the processor combines data from one or more sources to generate a plurality of curves and/or markers to be plotted on a graph, such as the graph illustrated in FIG. 12. The processor then sends data representative of that graph to a display. This step may be performed in response to a user command, such as a command received in step 1433, or may be performed continuously in a loop to update a displayed graph in real time.

The data can be processed in many ways. For example, time to close an incision can be determined from the data. The time between suture dispense from the dispensing unit such as a suture pack and placement in the needle receptacle can be used to determine speed of the surgeon or other user wearing the barrier. Moreover, speed may be further divided into a time from dispensing a need until securing that needle or a time from securing a needle until a dispensing a new needle.

Although FIG. 14 shows a method of capturing data in accordance with an example, a person of ordinary skill in the art will recognize many variations. The steps can be performed in any order, and steps can be added, repeated or deleted. The steps can be performed in any order. Some of the steps may comprise sub-steps, and some of the steps may be repeated more often than other steps.

A principal concern for infection control in the surgical environment are the people, tools, and supplies working within the immediate surgical, but how these factors arrive to the operating room can be of equal importance. A carefully orchestrated workflow can be key to minimizing the risk of contamination in this cleanest of patient care environments. Anything that moves in and out of the operating rooms, as well as the surgical suite as a whole, should be subject to rigorous control. Moreover, moisture vectors in this environment should be aggressively controlled.

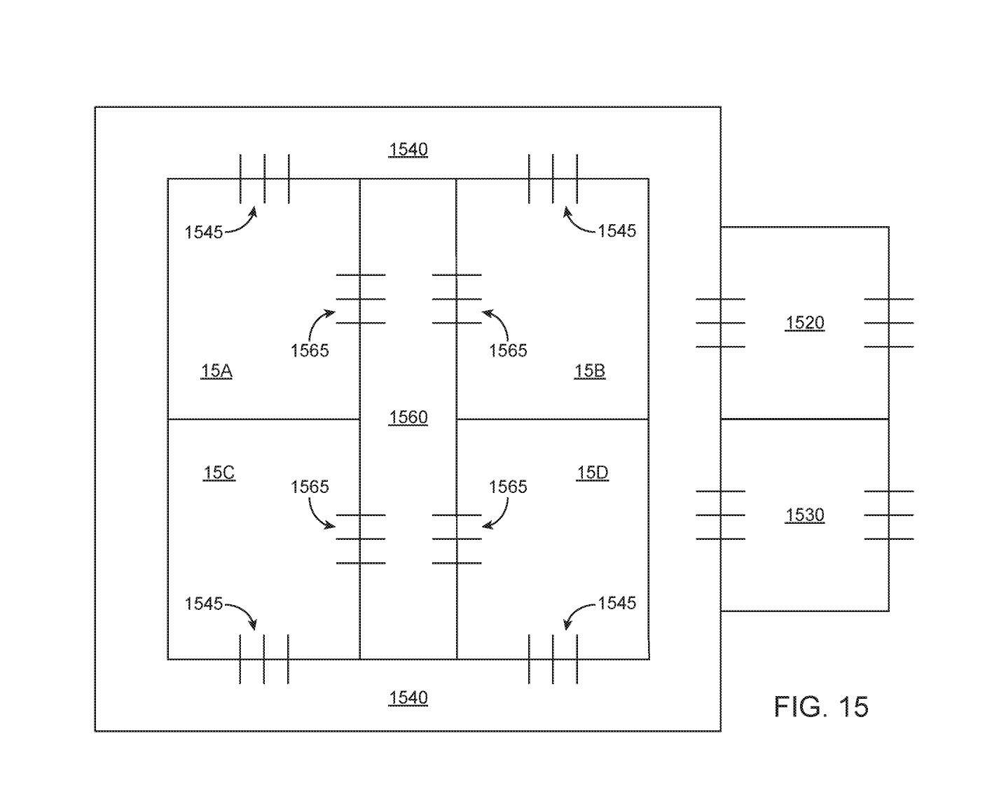

There are many configurations of surgery design in use, each with their own strengths and weaknesses. For example, double-loaded corridors with sub-sterile rooms may not provide optimal opportunity to prevent contamination or infection transfer as the mixed use of the shared corridor for people, patients, sterile materials and bio-hazardous waste poses a risk of infection. Another design is a perimeter corridor with a clean core, as shown in FIG. 15.

FIG. 15 illustrates a surgery design configuration, in accordance with an example embodiment. As shown in FIG. 15, sterile operating room suites 15A-D are linked by internal sterile core 1560 connecting the suites. One or more sterile supply areas (not pictured) may also be accessible from the core. Each operating room has a first door 1565 to the sterile core for use by, for example, circulating nurses when retrieving sterile instruments, or supplies. Note that each door may be bifurcated into two one-way portals with respective sensors or scanners. The operating rooms are surrounded on the perimeter by a corridor 1540. This corridor may be sub sterile. Each operating room may have a second door 1545 to the perimeter or sub sterile corridor 1540. Pre-op 1530 and soiled utility/clean-up 1520 rooms are provided separate from the operating rooms and clean core. These room are accessible from the operating rooms through the perimeter but not through the core.

Generally, only instruments, supplies, and non-moisture-based reprocessing units should be within the core. Surgical equipment should not be placed within the clean core, as it tends to move between operating rooms and thus increases risk of contamination if moved in and out of the core. Wipe-down cleaning by staff, although mandatory, may not render the equipment suitable for holding in the clean core with sterile surgical supplies.

A one-way flow of supplies into the operating room, then of soiled goods and trash out of the operating room, may be preferred. The shared use of a corridor for staff and patient access into the operating room can be acceptable, but this same corridor may not be used for delivery of sterile supplies into the operating room. Sterile supplies and instruments may have a separate, dedicated pathway from central sterile supply (e.g. accessible from the sterile core 1560) into the operating room without encountering staff or patient traffic or not.

Various embodiments of the present invention can monitor and optimize operating room workflow to enforces such operational norms and mitigate risk of infection. For example, various sensors can detect the coming and going from the operating room of personnel, instruments, equipment, and supplies, for example, through doors 1545 and 1565. These items may be recognized through optical object recognition, machine readable codes, color codes, RFID, etc. Multiple types of detection devices such as cameras, badge scanners, may be placed throughout the surgical suite, for example at doors, or other boundaries between rooms or sterile zones. Alarms or alerts may be presented when one of the operational norms has been violated or is close to being violated during a surgical procedure. Reports may also be generated indicating a performance of the surgical team based on a number or degree of violations.

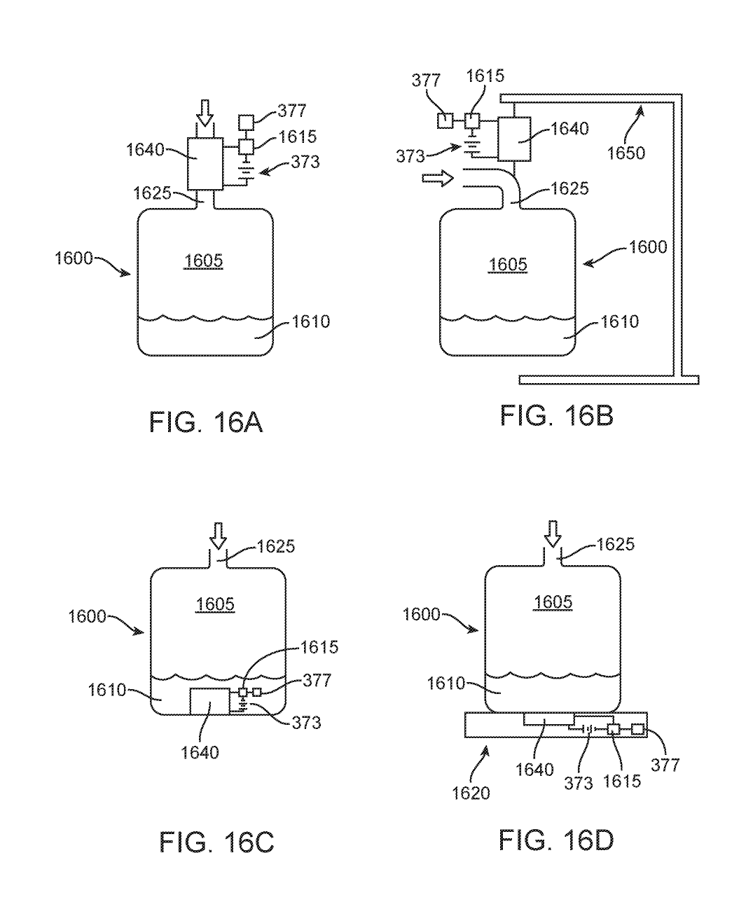

FIGS. 16A-16D schematically illustrate exemplary embodiments of urine storage systems for the tracking of urine volume from a patient through time. The urine storage systems as described herein may comprise a urine storage vessel 1600 with (see FIGS. 16A, 16B, and 16C) or without (see FIG. 16D) integral urine volume sensing capabilities. In embodiments where the urine storage system is without integral urine volume sensing capabilities, an external scale 1620 may be provided. In many embodiments, urine storage vessel 1600 may comprise an internal volume 1605 appropriate for the surgical procedure being performed, and be made of suitable material for storing urine 1610 from the patient. In some embodiments, urine storage vessel 1600 comprises a bag with internal volume 1605. In some embodiments, urine storage vessel 1600 comprises a vessel with a defined internal cross-sectional area and height (e.g., a cylinder with rigid walls) forming internal volume 1605. In some embodiments, urine storage vessel sponge 1600 may comprise transparent or semi-transparent vessel walls with integral or external indicators of the volume of urine 1610 therein that can provide a visual indication of the volume of urine stored through time. In many embodiments, urine storage vessel 1600 and/or scale 1620 may comprise sensing and control circuitry 1630 to provide urine volume sensing capabilities. The sensing and control circuitry 1630 may comprise a sensor 1640, control circuitry 1615, visual display 377, and power source 377 (power source 377 may comprise a battery, such as a lithium ion battery or any other suitable electrical power source).

In some embodiments, sensor 1640 may comprise a flowmeter disposed at an inlet 1625 of urine storage vessel 1600, the sensor configured to track the volume of urine 1610 from the patient through time (see FIG. 16A). In some embodiments, sensor 1640 may comprise a pressure transducer disposed between the urine storage vessel 1600 and a holder 1650 configured to support the urine storage vessel 1600 in a hanging configuration, the sensor configured to sense the weight of the urine storage vessel through time and thereby calculate the volume of urine 1610 from the patient through time (see FIG. 16B). In some embodiments, sensor 1640 may comprise a pressure transducer disposed within and at the bottom of the urine storage vessel 1600, the sensor configured to sense the pressure exerted by urine 1610 stored in the urine storage vessel 1610 above the sensor through time and thereby calculate the volume of urine 1610 from the patient through time (i.e., given the defined internal cross-sectional area of the urine storage vessel, the volume can be calculated as the internal cross-sectional area times the height, where the height is determined as the pressure exerted on the sensor divided by the specific gravity of urine (e.g., between 1.000 and 1.030), divided by the force of gravity; see FIG. 16C).

Whether configured to be integral with urine storage vessel 1600 or scale 1620, the sensing and control circuitry 1615 can output a signal to the visual display 377 that corresponds to the volume of urine 1610 stored in urine storage vessel 1600. In many embodiments, control circuitry 1615 can track the volume of urine stored in urine storage vessel 1600 through time. In an embodiment, visual display 377 can display the total volume of urine stored in absolute volume (e.g., mL of urine stored). In other embodiments, the display 377 can output any other visual indication of the volume of urine stored. Additional circuitry, such as wireless communication circuitry, can be coupled to the sensing and control circuitry 1615 of urine storage vessel 1600 and/or scale 1620 to track the volume of urine stored, and to time stamp and transmit this data as described herein.

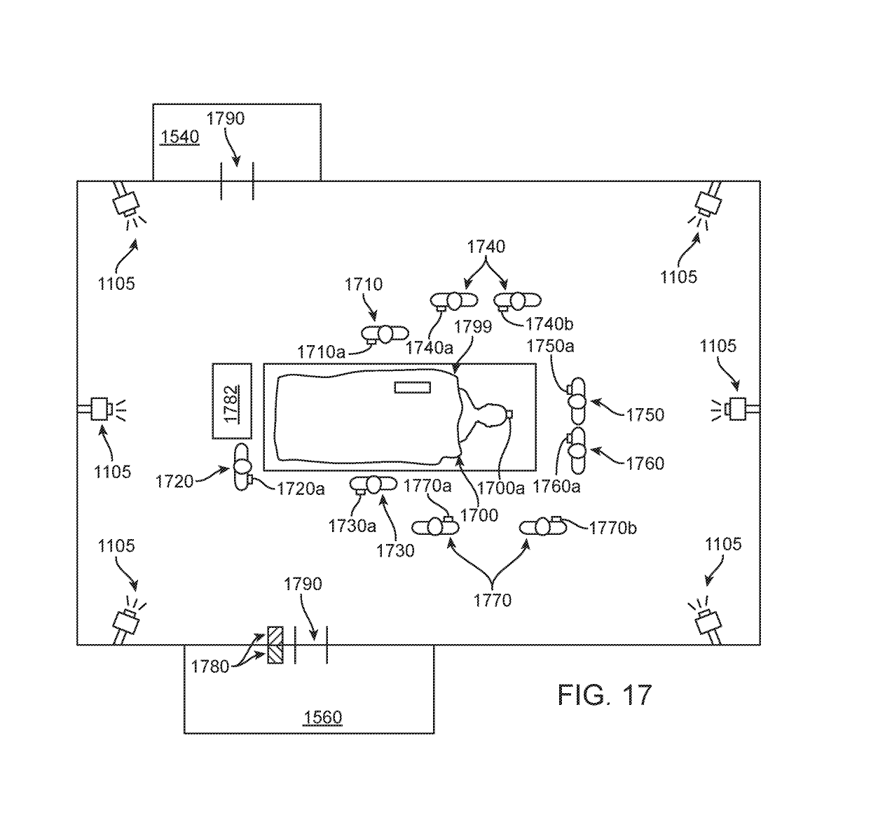

FIG. 17 schematically illustrates an example of the personnel involved in an operating room procedure (shown in a top view). Operating room personnel may comprise a surgeon 1710, a scrub technician 1720, an assistant surgeon 1730, one or more residents 1740, an anesthesiologist 1750, an anesthesia technician 1760, and one or more nurses 1770 (including, for example, a circulating nurse and a turnover nurse) in addition to patient 1700 in an operating room 1790. In many procedures, the surgeon 1710, scrub technician 1720, assistant surgeon 1730, and the one or more residents may be scrubbed in (i.e., have gone through sterilization procedures for surgery and don sterile attire) for the operating room procedure. The scrub tech may provide the surgeon, assistant surgeon, resident, or anyone else performing surgery with instruments from the surgical instrument table 1782.

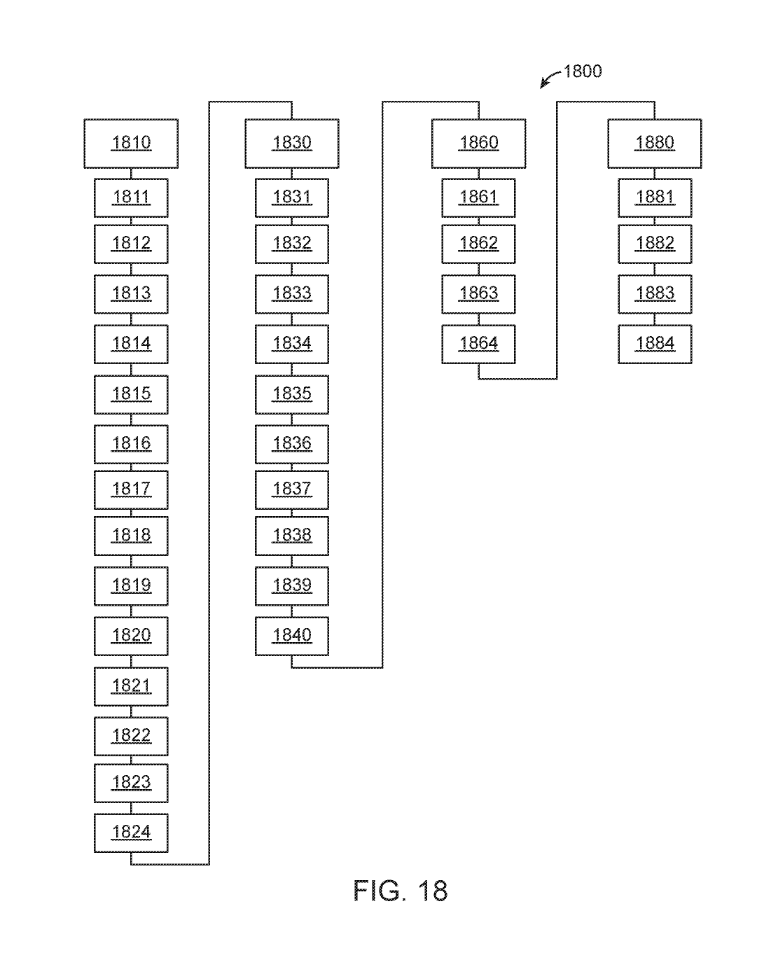

The location of operating room personnel may be tracked through time with a tracking system. The tracking system may be operably coupled with processor 160, display device 170, and database 190 as described herein to communicate and store tracking information through time. The information provided by the tracking system may be used to determine the presence or absence of operating room personnel during time intervals, including critical time intervals, associated with actions/procedures/steps as described herein (see, for example, FIG. 18 herein). In some embodiments, the tracking system may comprise unique identifiers 1700a, 1710a, 1720a, 1730a, 1740a, 1740b, 1750a, 1750b, 1770a, and 1770b associated with each of the personnel involved in a given operating room procedure (including the patient), and one or more scanners 1780 configured to register the entrance/exit of each person with the unique identifier to/from the operating room through an operating room entrance/exit 1790. For example, each of the personnel involved in an operating room procedure may be given a badge comprising a unique RFID chip, and scanner(s) at the entrance/exit of the operating room can register the entrance/exit of the personnel (and thereby their presence or absence in the room through time) via reading of the RFID signal. In some embodiments, the tracking system may comprise a biometric tracking system, wherein a picture, fingerprint, voice, or other biometric data may be used upon a person entering/exiting the operating room to track the location of the person through time. In some embodiments, the tracking system may be configured to track movement of operating room personnel throughout the hospital/clinic (e.g. by the tracking systems described herein or by GPS tracking).

In some embodiments, the motions of operating room personnel may be tracked. To enable monitoring and tracking of motions performed by operating room personnel, recording devices may be provided throughout the surgical environment of the operating room. One or more cameras 1105 disposed about the operating room may provide continuous video recording of the surgical procedure. Such cameras may, for example, be mounted on operating room walls, or on movable stands, allowing the cameras to be disposed at locations to conveniently capture video of the surgical procedure and motions being performed by the operating room personnel. The cameras may also incorporate lighting to illuminate their fields of view. In some embodiments, the recording devices may be configured to track arm and hand movements, akin to the tracking capabilities of currently available motion tracking systems (e.g. XBOX Kinect). In some embodiments, the movement or lack thereof of operating room personnel may be used to determine parameters such as surgeon efficiency, time intervals wherein a surgeon is waiting for devices and/or needles to be received from other operating room personnel, which personnel are performing a given task, and the like.

FIG. 18 illustrates an exemplary work flow for an operating room procedure through time. As shown, an operating room procedure 1800 may be divided into four primary time intervals, wherein each primary time interval may comprise various steps that form sub-intervals that may or may not be performed for each operating room procedure. The primary time intervals may comprise a time interval 1810 from when a patient first enters the operating room until a first incision into the patient is made by a surgeon, a time interval 1830 from when the first incision into the patient is made by the surgeon until a last incision into the patient is closed, a time interval 1860 from when the last incision into the patient is closed until the patient is moved out of the operating room, and a time interval 1880 from when the patient is moved out of the operating room until a next patient enters the operating room. In some embodiments, the last incision made into the patient comprises the first incision made into the patient, and closing of the last incision comprises closing the first incision.

The time interval 1810, from when the patient first enters the operating room until the first incision into the patient is made by the surgeon, may comprise the following steps: 1811, wherein the patient is brought into the operating room; 1812, wherein the patient is transferred from their bed to the operating room table; 1813, wherein the patient is positioned appropriately for the surgical procedure to be performed; 1814, wherein an oxygen dispenser is placed onto the patient; 1815, wherein the patient receives anesthesia; 1816, wherein an IV is placed into the patient; 1817, wherein a blood pressure monitor is placed into/onto the patient; 1818, wherein a pulse oximeter is placed onto a patient; 1819, wherein an EEG device is placed onto the patient; 1820, wherein an EKG device is placed into/onto the patient; 1821, wherein a respiration monitor is placed into/onto the patient; 1822, wherein a thermometer is placed into/onto the patient; 1823, wherein the patient is prepped and sterilized for the surgical procedure; and 1824, wherein the patient is draped 1799 for the surgical procedure.

The time interval 1830, from when the first incision into the patient is made by the surgeon until the last incision into the patient is closed, may comprise the following steps: 1831, wherein the first incision into the patient is made; 1832, wherein the surgical procedure is performed on the patient (e.g., transplant or device placed); 1833, wherein sponges are dispensed and used to absorb blood; 1834, wherein a surgical laser is used on the patient; 1835, wherein an ultrasonic probe is used on a patient; 1836, wherein a radiation therapy device is used on a patient; 1837, wherein an X-ray is performed on a patient; 1838, wherein fluoroscopy is performed on a patient; 1839, wherein an electrocautery device is used on a patient; and 1840, wherein a suture needle is dispensed from a needle dispensing unit, the suture is used on the patient, and the suture needle is stored in a needle receptacle.

The time interval 1860, from when the last incision into the patient is closed until the patient is moved out of the operating room, may comprise the following steps: 1861, wherein the last incision into the patient is closed; 1862, wherein anesthesia is halted; 1863, wherein external devices are removed from the patient (e.g., catheter, EEG device); and 1864, wherein the patient is transferred from the operating room table to a bed.

The time interval 1880, from when the patient is moved out of the operating room until the next patient enters the operating room, may comprise the following steps: 1881, wherein the patient is moved out of the operating room table; 1882, wherein consumables (e.g., sponges, drapes, suture needles) are restocked; 1883, wherein the operating room is sterilized; and 1884, wherein devices are replaced and/or sterilized.

Although FIG. 18 shows an exemplary workflow for an operating room procedure through time, a person of ordinary skill in the art will recognize many variations. The steps and their associated sub-intervals can be performed in any order, and steps can be added, repeated or deleted. In particular, steps corresponding to the workflow steps of FIG. 13, FIG. 14, and others described herein may be added as necessary. Some of the steps may comprise further steps with associated sub-intervals, and some of the steps may be repeated more often than other steps. Operating room procedure 1800 may be repeated by as necessary to accommodate multiple patients in any given day by cycling the time intervals/sub-steps described herein. Furthermore, some of the steps and their associated sub-intervals may be deemed critical, wherein the presence of the surgeon in the operating room is required. In particular, steps and associated sub-intervals within time intervals 1830 through 1840 may be deemed critical.

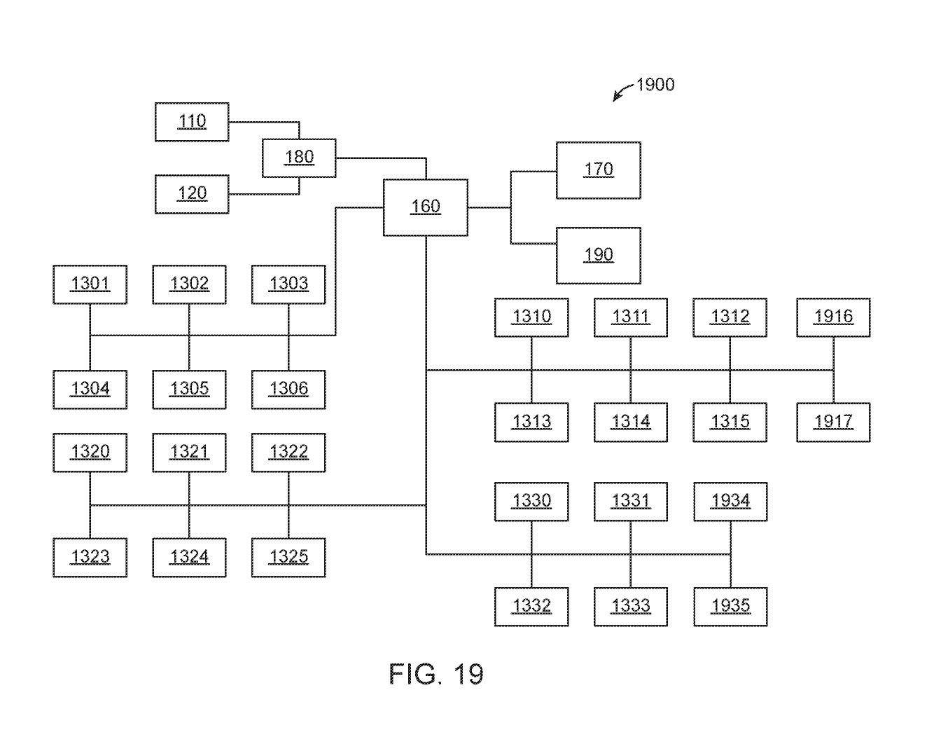

FIG. 19 illustrates a schematic diagram of an exemplary system 1900 for surgical workflow monitoring, in accordance with embodiments. As illustrated in FIGS. 2A and 2B herein, the dispensing and securing of needles by needle dispensing unit 110 and needle receptacle 120, respectively, as well as the usage of tools from tool receptacles may be tracked as a function of time by one or more sensors 180, which can communicate the tracking information to a processor 160. Further devices and modules may also be connected to the processor, each providing data to be processed. For example, electrocautery device 1301 may measure quantities such as voltage and current so as to determine electrical power usage as a function of time, and communicate these values to the processor 160. X-ray device 1302 may record and transmit the timing of X-ray images, as well as an associated intensity and/or pulse duration. Fluoroscope 1303 may record and transmit data representative of radiation intensity as a function of time. Ultrasonic probe 1304 may record and transmit data representative of ultrasonic pulse duration and intensity as a function of time. Surgical laser 1305 may record and transmit data representative of laser pulse duration and intensity as a function of time. Radiation therapy device 1306 may record and transmit data representative of the amount of radiation applied as a function of time. Further devices may be used to assess quantities of fluids and gases to/from the patient as a function of time. Anesthesia administering device 1310 may measure the amount of each anesthetic administered to the patient as a function of time, for example, by recording and transmitting flow rates for each anesthetic as a function of time. Blood transfusion device 1311 may measure the amount of blood administered to the patient as a function of time, for example, by recording and transmitting flow rates from a source of blood for transfusion. Further devices that may be used include devices to administer antibiotics 1312, anticoagulants 1313, and other intravenous fluid dispensers 1314. Each such device may monitor its respective flow as a function of time and transmits corresponding data to the processor 160. Gas flow to the patient may be monitored and transmitted to the processor 160 as a function of time, for example by oxygen dispenser 1315. Generally, gas and liquid flow may be measured by devices such as mechanical or electrical flowmeters or by monitoring total remaining fluid as a function of time. Gas flow as a function of time may also be measured in other ways, such as monitoring gas pressure across a regulator to estimate flow. Sponges 1916 as described in FIGS. 15A-E or blood suction devices may measure the amount of blood absorbed/withdrawn by each as a function of time (and thereby serve as a measure of the blood lost by the patient as a function of time). Urine storage systems 1917 as described in FIGS. 16A-D may measure the amount of urine stored from the patient as a function of time.

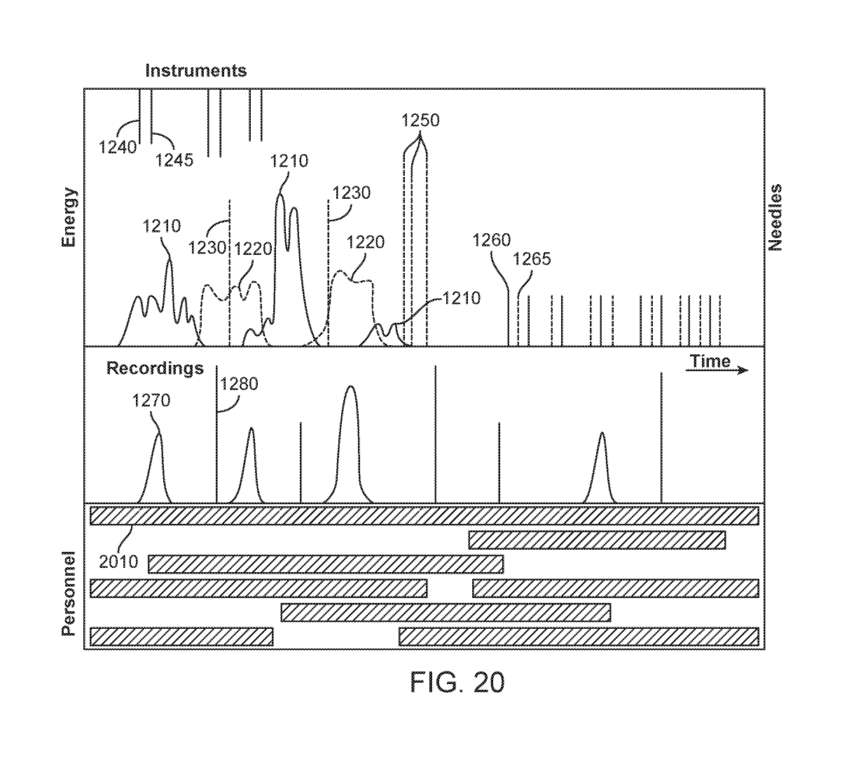

Patient vital signs may be monitored as appropriate during a surgical procedure as a function of time. Monitoring devices include blood pressure meter 1320, pulse oximeter 1321, EEG device 1322, EKG device 1323, respiration monitor 1324, and thermometer 1325. Each device may record continuous data over time and transmits its readings to the processor 160. Further recording devices, such as audio recorders 1330, video cameras 1331, digital cameras 1332, and mobile devices 1333 record audio, video, still images, or a combination thereof, can transmit corresponding data to the processor 160 as a function of time. Location tracking 1934 and movement tracking 1935 of operating room personnel may be recorded as a function of time as described herein, and transmitted to the processor 160. The processor 160 may receive data from each connected device and record corresponding information in memory as a function of time, such as in database 190. The processor 160 may also produce graphical representations of the recorded data, such as those shown in FIG. 20, for display in display device 170. Display device 170 may, for example, be a mobile device in the operating room, such as mobile device 1220, allowing real time display of relevant data during a surgical procedure. Display device 170 may also, for example, be a mobile device at the front desk, such as mobile device 1220, allowing real time display of relevant data across operating rooms during surgical procedures.

As disclosed in further detail herein, each connection to the processor may independently be wired or wireless, as needed to ensure reliability and accuracy of data transmission. The data may also be transmitted in a secure format, for example, using data encryption, to protect confidentiality. Time may also continuously be tracked for each device connected to the processor, either locally using a synchronized clock, or globally by a clock associated with the processor 160, or both. For devices using the processor clock, timing information may be based on the time data as received by the processor. For locally timed devices, timestamps or other timing data may be transmitted along with signal data.

The recorded data as described herein may be used to provide a comprehensive understanding of surgical procedures. For example, FIG. 20 shows a graphical representation of data that may be recorded during a surgical procedure. The data can be recorded with appropriate time stamps that are registered to provide the output graph with a common time base. The data recorded are illustrated as a function of time, with the top panel representing both the use of surgical tools and the application of energy in various forms to the patient during the surgical procedure, the middle panel representing recordings of video and audio, and the lower panel representing the presence and absence of operating personnel in the operating room. The top panel of FIG. 20 shows curves representing the amount of energy applied per unit time for surgical procedures such as electrocautery 1210, fluoroscopy 1220, and X-ray imaging 1230. Each curve may be generated by a monitoring process such as that described above for electrocautery, and the recorded data may be collected by a central server connected to devices such a electrocautery machines, fluoroscopes, and X-ray machines, each of which may monitor its own amount of power applied to the patient. Each curve may be integrated to determine a quantity proportional to its total respective energy applied by each source. The recording and display of each curve may be individually optional, and additional curves may be recorded for further devices that may apply energy to the patient, such as surgical lasers, ultrasound probes, electromagnetic radiation or particle pulses, etc. In the case of short pulses such as X-rays, which are typically recorded briefly and are shown as sharp peaks in FIG. 20, a single value may in some cases be recorded for each peak, representing its respective total energy, such as X-ray energy, applied. Each type of energy signal may be recorded separately, but in some cases may be combined; for example, the total amount of X-ray and fluoroscope usage may be integrated together using appropriate weighting parameters to determine a total exposure to ionizing radiation from these sources. In addition to energy measurements, other metrics may be recorded during the surgical procedure. For example, flow of anesthesia, transfused blood, antibiotics, anticoagulants, and other intravenous fluids into the patient may be recorded by flowmeters. Gas input such as oxygen may be similarly recorded. Patient vital indicators may also be monitored and recorded, such as blood oxygenation, pulse, electroencephalogram (EEG), electrocardiogram (EKG), respiration, body temperature, and blood pressure. Blood lost from the patient (absorbed by sponges or suctioned away) may be monitored and recorded. Urine from the patient may be monitored and recorded. Light level, temperature, humidity and other qualities of the operating room environment may be recorded. Additionally shown in the top panel is tool usage, as monitored by the tool tracking system described herein. Each removal 1240 and replacement 1245 of each tool from tool receptacles may be recorded, with different tools represented by lines of different length. Additionally, the implanting of objects such as catheters, pacemakers, artificial or transplanted organs, artificial joints, surgical pins, rods, screws, or plates, etc. may be monitored and recorded, as shown by implant lines 1250. In some cases, the insertion of implants may be determined from video or X-ray images. Alternatively or additionally, the surgeon or surgical staff may indicate the times at which each implant is inserted, for example, by speech, actuation of a lever, pressing of a button, etc. At the end of the procedure, the surgeon closes using a plurality of needles to suture the patient. Each time a needle is dispensed 1260 or secured 1265, the system records that fact as well as the time it happens, allowing a graphical representation of exactly how much time is spent between each suture added to the patient. This can help in understanding surgeon workflow and give insight into how to optimize surgical procedures and cut down on wasted time. Data from different surgeons may be compared, as well as data from the same surgeon performing either similar or dissimilar procedures, allowing trends to be detected; for example, surgical efficiency may vary depending on time of day, day of the week, time between operations, total time spent so far in a shift, etc. Correlations may also be found between, for example, patient vital signs and particular steps of surgical procedures; for example, the use of certain tools may correlate with changes in blood pressure or oxygenation, either because their use causes the changes, or because they are used in response to such changes.

The middle panel of FIG. 20 shows a parallel recording of video and audio data that may be produced during a surgical procedure, illustrated on the same time axis as the top and bottom panels. The audio recordings 1270 are shown, indicating total audio volume for each time period that recording is turned on. The audio recordings may be played back at a later time, for example, to review notes made by the surgeon and/or surgical staff during the procedure. Video may also be viewed of the procedure, along with or without the associated audio recordings. Images recorded by the surgeon or surgical staff are also shown by image markers 1280; these images may for example be snapshots of important moments in the surgical procedure. In some cases, the audio recording may include description of the images before, after, or during the time that the images are recorded. All of the data shown here may be combined by appropriate computing systems, and recorded, for example, in a database which may be accessed after the procedure for review and analysis. The data may be made available on a mobile device, such as a tablet or smartphone. The data may even be made available in real time during a surgical procedure; for example, on mobile device 1120. In order to allow the combination of all data inputs, each input device may be synchronized to a common time base, so that, as shown in FIG. 20, each marker line and curve may be associated with a particular time or period of time.

The bottom panel of FIG. 20 shows a parallel recording of the absence and presence of the operating room personnel in the operating room during a surgical procedure, illustrated on the same time axis as the top and middle panels. The presence of operating room personnel may be shown as horizontal bars 2010 through time; whereas the absence of operating room personnel may be shown as empty space. Alternatively, the presence of operating room personnel may be shown in any other binary means (e.g., a horizontal line that steps up for present, steps down for absent). However displayed, the presence/absence of each operating room personnel (including the patient) is represented by a single row, thus as shown FIG. 20 displays the absence/presence of 6 operating room personnel through time. The lower panel may be configured to display as many horizontal rows, each representing one individual, as necessary to provide information on any and all individuals that have entered the operating room during a given surgical procedure. All of the data shown here may be combined by appropriate computing systems, and recorded, for example, in a database which may be accessed after the procedure for review and analysis. The data may be made available on a mobile device, such as a tablet or smartphone. The data may even be made available in real time during a surgical procedure; for example, on mobile device 1120. In order to allow the combination of all data inputs, each input device may be synchronized to a common time base, so that, as shown in FIG. 20, each representation of an individuals' absence or presence may be associated with a particular time or period of time.

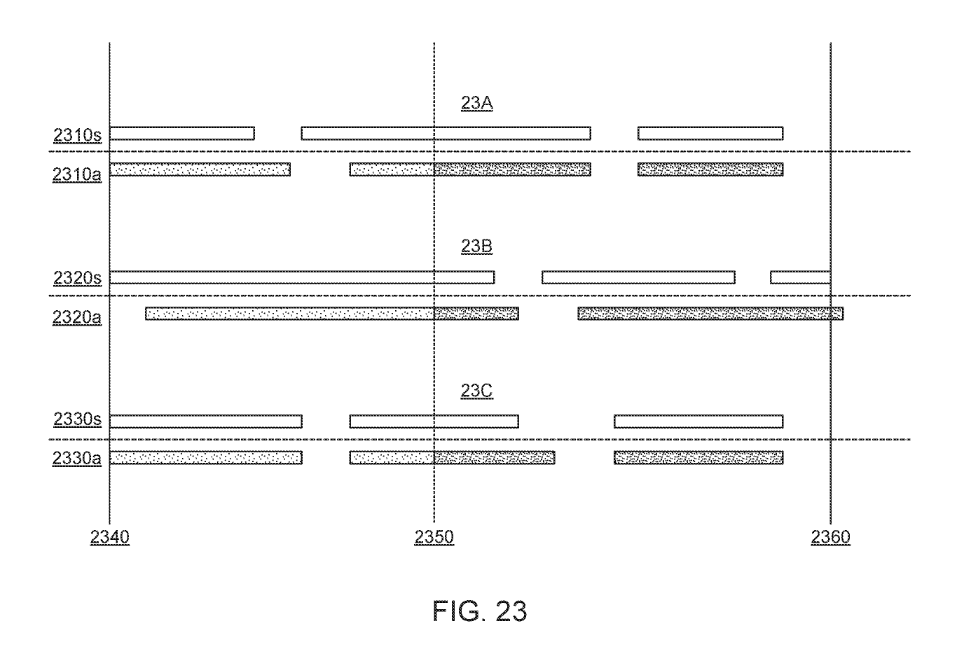

FIG. 23 shows a graphical representation of operating room status, in accordance with embodiments. A control room or operations center for managing a plurality of operating rooms may maintain a graphical representation of surgical procedures assigned to operating rooms over the day or another time period. As shown in FIG. 23, the graphical representation may include Gantt-style chart time bars 2310s, 2320s, 2330s representing scheduled surgical procedures in operating rooms 23A-C during a day between an opening time 2340 of a suite of operating rooms and a scheduled closing time 2360 of the suite of operating rooms. However, surgical procedures may start late, end early, be canceled, be moved to another operating room etc., so that the actual use of the operating rooms no longer track the initial expected use. In FIG. 23, time bars 2310a 2320s 2330a left of a current time 2350 represent the actual use of the operating rooms up until the current time. As described herein, the ending time for a surgical procedure may be estimated and updated in real time even during the surgical procedure, for example, based on the historical performance of the surgical team completing the operation or a current stage or status of the surgical procedure. As shown in FIG. 23, time bars 2310a 2320a 2330a after (right of) the current time 2350 represent up-to-date estimates of when surgical procedures should end and start. In some embodiments, the graphical representation may be updated in real-time to reflect estimated or predicted deviations from an operating room schedule.

As shown in FIG. 23, the scheduled surgical procedures for operating room 23B are now predicted t run past the closing time 2360. After a closing time of a suite of operating rooms, the cost of operating such a room may increase dramatically because of overtime pay and other factors. Thus, some embodiments may automatically reassign personnel, instruments, equipment, or operating rooms to schedule surgical procedures in order to achieve a configuration where the surgical procedure is predicted to end before closing time. Further embodiments may maximize the use of a suite of operating room for surgical procedures while minimizing non-used time of operating rooms between an opening and closing of the suite of operating rooms.

The graphical representations described herein can be shown on a display of a user device. The graphical display can be interactive and allow the user to obtain additional detail on each of the structures of the report. The structure of the report may comprise one or more items shown on graphical representation the display, such as an image. The user may touch on one of the items to view additional detail, for example by touching an appropriate item on a touch screen display.

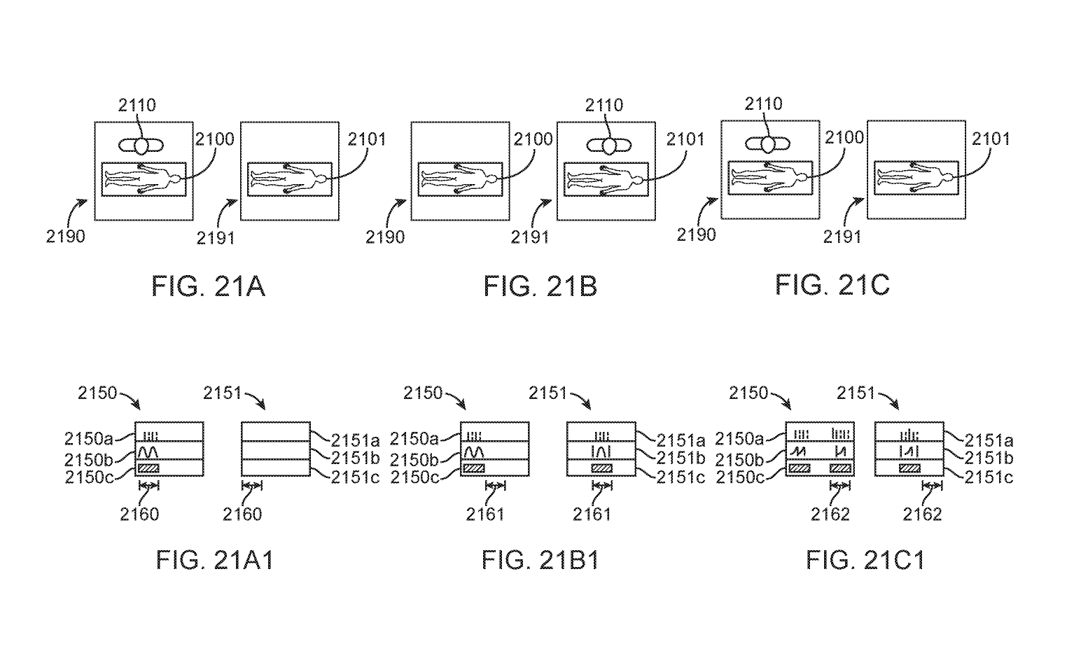

It is acceptable practice for a surgeon to operate in one or more operating rooms concurrently, as long as the surgeon is present for the critical procedures of each. The information and tracking systems provided herein may be configured to track, record, and report such circumstances, wherein the surgeon moves from one operating room to at least one other. FIG. 21A illustrates an exemplary embodiment wherein a surgeon 2110 is operating in a first operating room 2190 and performing critical procedures as defined herein on a first patient 2100, while a second patient 2101 in a second operating room 2191 has been staged appropriately. After completing the critical procedures in operating room 2190, the surgeon may scrub out, leave the first operating room 2190, scrub into the second operating room 2191, and perform critical procedures on the second patient 2101 as shown in FIG. 21B. Next, as shown in FIG. 21C, the surgeon 2110 may scrub out of the second operating room 2191, scrub into the first operating room 2190, and perform any additional critical procedures on the first patient 2100.

Associated with the movement and procedures performed by surgeon 2110 exemplified in FIGS. 21A-C are graphical representations shown in FIGS. 21A1-C1. As shown, graphical representations, such as the graphical representation described in FIG. 20 herein, may be provided for each operating room (although simplified here for explanation purposes). In this example, FIG. 21A1 shows a first graphical representation 2150 corresponding to the information tracked in the first operating room 2190 during a time interval 2160, and a second graphical representation 2151 corresponding to the information tracked in the second operating room 2191 during the time interval 2160. Information such as instrument usage, energy usage by various devices, and needle usage (as described in FIG. 20) may be provided in the top panels 2150a and 2151a of the graphical representations; information such as audio, video, and digital recordings (as described in FIG. 20) may be provided in the middle panels 2150b and 2151b of the graphical representations; and information on the presence and absence of personnel (as described in FIG. 20, in this case only the surgeon is shown) may be provided in the bottom panels 2150c and 2151c of the graphical representations. In this example, the movement and procedures of the surgeon 2110 between the first and second operating rooms are continually tracked through time and represented in graphical representations 2150 and 2151 in FIG. 21B1 and FIG. 21C1, corresponding to FIG. 21B and FIG. 21C, respectively. In this way, reports can be generated and the absence and presence of a surgeon during critical procedures verified.

The scenario described in FIGS. 21A-C and FIGS. 21A1-C1 is by way of example; any number of operating rooms may be staged such that a surgeon can perform procedures, including critical procedures, between different operating rooms at different times. Furthermore, the surgeon may be return to an operating room to perform additional procedures if necessary after performing a first set of procedures therein. The information and tracking systems described herein can track, record, and report all and such circumstances.

The detailed surgical workflow and timing information as described herein sent to processor 160 and stored in database 190 may be analyzed by a program provided to the processor 160 and configured to output numerous parameters of interest. Parameters of interest may include operating room personnel efficiency, a grade of operating room personnel performance, synergies between operating room personnel, and statistics that can be used for predictive analytics and scheduling of future operating room cases/procedures. For example, scheduling of future operating room cases/procedures may be based on the most efficient working team that can be provided given personnel availability, personnel efficiency, personnel synergies, patient information, case/procedure complexity, and the like. Importantly, the disclosure provided herein may provide quantitative data, that is actionable, to an environment wherein qualitative data is currently provided.

### Certain Definitions

Unless otherwise defined, all technical terms used herein have the same meaning as commonly understood by one of ordinary skill in the art to which this invention belongs. As used in this specification and the appended claims, the singular forms “a,” “an,” and “the” include plural references unless the context clearly dictates otherwise. Any reference to “or” herein is intended to encompass “and/or” unless otherwise stated.

### Digital Processing Device

In some embodiments, the platforms, systems, media, and methods described herein include a digital processing device, or use of the same. In further embodiments, the digital processing device includes one or more hardware central processing units (CPUs) or general purpose graphics processing units (GPGPUs) that carry out the device's functions. In still further embodiments, the digital processing device further comprises an operating system configured to perform executable instructions. In some embodiments, the digital processing device is optionally connected a computer network. In further embodiments, the digital processing device is optionally connected to the Internet such that it accesses the World Wide Web. In still further embodiments, the digital processing device is optionally connected to a cloud computing infrastructure. In other embodiments, the digital processing device is optionally connected to an intranet. In other embodiments, the digital processing device is optionally connected to a data storage device.

In accordance with the description herein, suitable digital processing devices include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Those of skill in the art will recognize that many smartphones are suitable for use in the system described herein. Those of skill in the art will also recognize that select televisions, video players, and digital music players with optional computer network connectivity are suitable for use in the system described herein. Suitable tablet computers include those with booklet, slate, and convertible configurations, known to those of skill in the art.

In some embodiments, the digital processing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smart phone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®. Those of skill in the art will also recognize that suitable media streaming device operating systems include, by way of non-limiting examples, Apple TV®, Roku®, Boxee®, Google TV®, Google Chromecast®, Amazon Fire®, and Samsung® HomeSync®. Those of skill in the art will also recognize that suitable video game console operating systems include, by way of non-limiting examples, Sony® PS3®, Sony® PS4®, Microsoft® Xbox 360®, Microsoft Xbox One, Nintendo® Wii®, Nintendo® Wii U®, and Ouya®.

In some embodiments, the device includes a storage and/or memory device. The storage and/or memory device is one or more physical apparatuses used to store data or programs on a temporary or permanent basis. In some embodiments, the device is volatile memory and requires power to maintain stored information. In some embodiments, the device is non-volatile memory and retains stored information when the digital processing device is not powered. In further embodiments, the non-volatile memory comprises flash memory. In some embodiments, the non-volatile memory comprises dynamic random-access memory (DRAM). In some embodiments, the non-volatile memory comprises ferroelectric random access memory (FRAM). In some embodiments, the non-volatile memory comprises phase-change random access memory (PRAM). In other embodiments, the device is a storage device including, by way of non-limiting examples, CD-ROMs, DVDs, flash memory devices, magnetic disk drives, magnetic tapes drives, optical disk drives, and cloud computing based storage. In further embodiments, the storage and/or memory device is a combination of devices such as those disclosed herein.

In some embodiments, the digital processing device includes a display to send visual information to a user. In some embodiments, the display is a cathode ray tube (CRT). In some embodiments, the display is a liquid crystal display (LCD). In further embodiments, the display is a thin film transistor liquid crystal display (TFT-LCD). In some embodiments, the display is an organic light emitting diode (OLED) display. In various further embodiments, on OLED display is a passive-matrix OLED (PMOLED) or active-matrix OLED (AMOLED) display. In some embodiments, the display is a plasma display. In other embodiments, the display is a video projector. In still further embodiments, the display is a combination of devices such as those disclosed herein.

In some embodiments, the digital processing device includes an input device to receive information from a user. In some embodiments, the input device is a keyboard. In some embodiments, the input device is a pointing device including, by way of non-limiting examples, a mouse, trackball, track pad, joystick, game controller, or stylus. In some embodiments, the input device is a touch screen or a multi-touch screen. In other embodiments, the input device is a microphone to capture voice or other sound input. In other embodiments, the input device is a video camera or other sensor to capture motion or visual input. In further embodiments, the input device is a Kinect, Leap Motion, or the like. In still further embodiments, the input device is a combination of devices such as those disclosed herein.

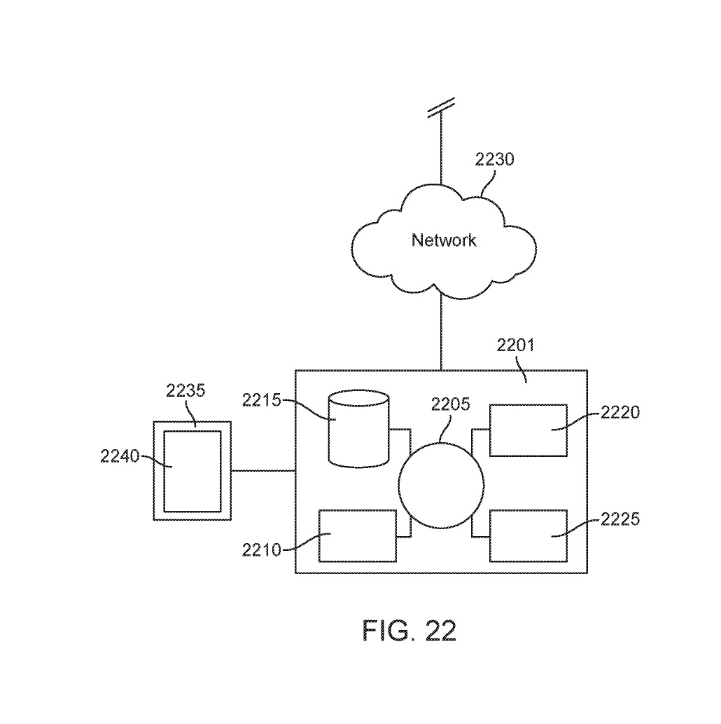

Referring to FIG. 22, in a particular embodiment, an exemplary digital processing device 2201 is programmed or otherwise configured to receive, generate, process, analyze, and output operating room information. In this embodiment, the digital processing device 2201 includes a central processing unit (CPU, also “processor” and “computer processor” herein) 2205, which can be a single core or multi core processor, or a plurality of processors for parallel processing. The digital processing device 2201 also includes memory or memory location 2210 (e.g., random-access memory, read-only memory, flash memory), electronic storage unit 2215 (e.g., hard disk), communication interface 2220 (e.g., network adapter) for communicating with one or more other systems, and peripheral devices 2225, such as cache, other memory, data storage and/or electronic display adapters. The memory 2210, storage unit 2215, interface 2220 and peripheral devices 2225 are in communication with the CPU 2205 through a communication bus (solid lines), such as a motherboard. The storage unit 2215 can be a data storage unit (or data repository) for storing data. The digital processing device 2201 can be operatively coupled to a computer network (“network”) 2230 with the aid of the communication interface 2220. The network 2230 can be the Internet, an internet and/or extranet, or an intranet and/or extranet that is in communication with the Internet. The network 2230 in some cases is a telecommunication and/or data network. The network 2230 can include one or more computer servers, which can enable distributed computing, such as cloud computing. The network 2230, in some cases with the aid of the device 2201, can implement a peer-to-peer network, which may enable devices coupled to the device 2201 to behave as a client or a server.

Continuing to refer to FIG. 22, the CPU 2205 can execute a sequence of machine-readable instructions, which can be embodied in a program or software. The instructions may be stored in a memory location, such as the memory 2210. The instructions can be directed to the CPU 2205, which can subsequently program or otherwise configure the CPU 2205 to implement methods of the present disclosure. Examples of operations performed by the CPU 2205 can include fetch, decode, execute, and write back. The CPU 2205 can be part of a circuit, such as an integrated circuit. One or more other components of the device 2201 can be included in the circuit. In some cases, the circuit is an application specific integrated circuit (ASIC) or a field programmable gate array (FPGA).

Continuing to refer to FIG. 22, the storage unit 2215 can store files, such as drivers, libraries and saved programs. The storage unit 2215 can store user data, e.g., user preferences and user programs. The digital processing device 2201 in some cases can include one or more additional data storage units that are external, such as located on a remote server that is in communication through an intranet or the Internet.

Continuing to refer to FIG. 22, the digital processing device 2201 can communicate with one or more remote computer systems through the network 2230. For instance, the device 2201 can communicate with a remote computer system of a user. Examples of remote computer systems include personal computers (e.g., portable PC), slate or tablet PCs (e.g., Apple® iPad, Samsung® Galaxy Tab), telephones, Smart phones (e.g., Apple® iPhone, Android-enabled device, Blackberry®), or personal digital assistants.

Methods as described herein can be implemented by way of machine (e.g., computer processor) executable code stored on an electronic storage location of the digital processing device 2201, such as, for example, on the memory 2210 or electronic storage unit 2215. The machine executable or machine readable code can be provided in the form of software. During use, the code can be executed by the processor 2205. In some cases, the code can be retrieved from the storage unit 2215 and stored on the memory 2210 for ready access by the processor 2205. In some situations, the electronic storage unit 2215 can be precluded, and machine-executable instructions are stored on memory 2210.

### Non-Transitory Computer Readable Storage Medium

In some embodiments, the platforms, systems, media, and methods disclosed herein include one or more non-transitory computer readable storage media encoded with a program including instructions executable by the operating system of an optionally networked digital processing device. In further embodiments, a computer readable storage medium is a tangible component of a digital processing device. In still further embodiments, a computer readable storage medium is optionally removable from a digital processing device. In some embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media.

### Computer Program

In some embodiments, the platforms, systems, media, and methods disclosed herein include at least one computer program, or use of the same. A computer program includes a sequence of instructions, executable in the digital processing device's CPU, written to perform a specified task. Computer readable instructions may be implemented as program modules, such as functions, objects, Application Programming Interfaces (APIs), data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft® .NET or Ruby on Rails (RoR). In some embodiments, a web application utilizes one or more database systems including, by way of non-limiting examples, relational, non-relational, object oriented, associative, and XML database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

### Mobile Application

In some embodiments, a computer program includes a mobile application provided to a mobile digital processing device. In some embodiments, the mobile application is provided to a mobile digital processing device at the time it is manufactured. In other embodiments, the mobile application is provided to a mobile digital processing device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Google® Play, Chrome WebStore, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation is often performed, at least in part, to create an executable program. In some embodiments, a computer program includes one or more executable complied applications.

### Web Browser Plug-In

In some embodiments, the computer program includes a web browser plug-in (e.g., extension, etc.). In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®. In some embodiments, the toolbar comprises one or more web browser extensions, add-ins, or add-ons. In some embodiments, the toolbar comprises one or more explorer bars, tool bands, or desk bands.

In view of the disclosure provided herein, those of skill in the art will recognize that several plug-in frameworks are available that enable development of plug-ins in various programming languages, including, by way of non-limiting examples, C++, Delphi, Java™, PHP, Python™, and VB .NET, or combinations thereof.

Web browsers (also called Internet browsers) are software applications, designed for use with network-connected digital processing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. In some embodiments, the web browser is a mobile web browser. Mobile web browsers (also called mircrobrowsers, mini-browsers, and wireless browsers) are designed for use on mobile digital processing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

### Software Modules

In some embodiments, the platforms, systems, media, and methods disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

### Databases

In some embodiments, the platforms, systems, media, and methods disclosed herein include one or more databases, or use of the same. In view of the disclosure provided herein, those of skill in the art will recognize that many databases are suitable for storage and retrieval of operating room information. In various embodiments, suitable databases include, by way of non-limiting examples, relational databases, non-relational databases, object oriented databases, object databases, entity-relationship model databases, associative databases, and XML databases. Further non-limiting examples include SQL, PostgreSQL, MySQL, Oracle, DB2, and Sybase. In some embodiments, a database is internet-based. In further embodiments, a database is web-based. In still further embodiments, a database is cloud computing-based. In other embodiments, a database is based on one or more local computer storage devices.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. An apparatus to measure surgical workflow, the apparatus comprising:
a processor configured with instructions to receive inputs corresponding to a plurality of surgical parameters related to surgery of a patient.

2. The apparatus of claim 1, wherein the plurality of inputs comprises a plurality of times corresponding to one or more of removal of needles from a suture pack or placement of needles in a needles receptacle.

3. The apparatus of claim 1, wherein the processor is configured to provide an alert when a first needle and a second needle have been removed from a suture pack without the first needle having been placed in a needle receptacle.

4. The apparatus of claim 1, wherein the processor is configured to provide an alert when a suture needle has been removed from a pack before the needle has been placed in a receptacle.

5. The apparatus of claim 2, wherein the plurality of inputs comprises a plurality of times at which each of a plurality of needles is removed from a suture pack.

6. The apparatus of claim 2, wherein the plurality of inputs comprises a plurality of times at which each of a plurality of needles is placed in a needle receptacle.

7. The apparatus of claim 2, wherein the plurality of inputs comprises a unique identifier from a suture pack.

8. The apparatus of claim 2, wherein the plurality of inputs comprises a plurality of unique identifiers from one or more of a plurality of suture packs or each of a plurality of needles.

9. The apparatus of claim 2, wherein the plurality of inputs comprises a plurality of unique identifiers from a plurality of needle receptacles.

10. The apparatus of claim 2, wherein the plurality of inputs comprises a plurality of unique identifiers from a plurality of suture packs and a plurality of unique identifiers from a plurality of needle receptacles and a plurality of times at which each of the plurality of needles is removed from a corresponding suture pack and a plurality of times at which each of the plurality of needles is placed in a corresponding needle receptacle.

11. The apparatus of claim 2, wherein the plurality of inputs comprises a unique identifier of a person wearing a surgical barrier.

12. The apparatus of claim 2, wherein the plurality of inputs comprises a unique identifier of a surgical barrier worn by a person during surgery.

13. The apparatus of claim 2, wherein the processor comprises instructions to register the plurality of times with a plurality of times from one or more of an optical image, a physician dictation, a video image, a smartphone image, a fluoroscopy radiation dosage, an x-ray radiation dosage from an x-ray, in instrument removal from a holder, an instrument placement into a holder, an electrocautery dosage from an electrocautery device, an implant time at which an implant is placed in the patient, an audio recording, or an image.

14. The apparatus of claim 2, wherein the processor comprises instructions to determine an amount of time to close surgical incision in response to the plurality of times.

15. The apparatus of claim 1, wherein the processor comprises instructions to generate a graph with a common time base for one or more of suture removal from a pack, suture placement in a suture receptacle, a video image, a physician dictation, a video image, a smartphone image, a fluoroscopy radiation dosage, an x-ray radiation dosage from an x-ray, in instrument removal from a holder, an instrument placement into a holder, an electrocautery dosage from an electrocautery device, an implant time at which an implant is placed in the patient, an audio recording, or an image.

16. The apparatus of claim 15, wherein said graph comprises an interactive data file in which a user can identify a structure of the graph and view additional detail of the structure.

17. The apparatus of claim 16, wherein the identified structure of the graph comprises information related to one or more of suture removal from a pack, suture placement in a suture receptacle, a video image, a physician dictation, a video image, a smartphone image, a fluoroscopy radiation dosage, an x-ray radiation dosage from an x-ray, in instrument removal from a holder, an instrument placement into a holder, an electrocautery dosage from an electrocautery device, an implant time at which an implant is placed in the patient, an audio recording, or an image.

18. The apparatus of claim 1, wherein the processor comprises a processor system.

19. An method to measure surgical workflow, the method comprising:
receiving with a processor inputs corresponding to a plurality of surgical parameters related to surgery of a patient.

20. The method of claim 19, wherein the processor provides an alert when a suture needle has been removed from a pack before the needle has been placed in a receptacle.

21. The method of claim 19, wherein the processor provides an alert when a first needle and a second needle have been removed from a suture pack without the first needle having been placed in a needle receptacle.

22. The method of claim 19, wherein the plurality of inputs comprises a plurality of times corresponding to one or more of removal of needles from a suture pack or placement of needles in a needles receptacle.

23. The method of claim 22, wherein the plurality of inputs comprises a plurality of times at which each of a plurality of needles is removed from a suture pack.

24. The method of claim 22, wherein the plurality of inputs comprises a plurality of times at which each of a plurality of needles is placed in a needle receptacle.

25. The method of claim 22, wherein the plurality of inputs comprises a unique identifier from a suture pack.

26. The method of claim 22, wherein the plurality of inputs comprises a plurality of unique identifiers from a plurality of suture packs.

27. The method of claim 22, wherein the plurality of inputs comprises a plurality of unique identifiers from a plurality of needle receptacles.

28. The method of claim 22, wherein the plurality of inputs comprises a plurality of unique identifiers from a plurality of suture packs and a plurality of unique identifiers from a plurality of needle receptacles and a plurality of times at which each of the plurality of needles is removed from a corresponding suture pack and a plurality of times at which each of the plurality of needles is placed in a corresponding needle receptacle.

29. The method of claim 22, further comprising registering the plurality of times with a plurality of times from one or more of an optical image, a physician dictation, a video image, a smartphone image, a fluoroscopy radiation dosage, an x-ray radiation dosage from an x-ray, in instrument removal from a holder, an instrument placement into a holder, an electrocautery dosage from an electrocautery device, an implant time at which an implant is placed in the patient, an audio recording, or an image.

30. The method of claim 22, further comprising determining an amount of time to close a surgical incision in response to the plurality of times.

31. The method of claim 19, further comprising generating a graph with a common time base for one or more of suture removal from a pack, suture placement in a suture receptacle, a video image, a physician dictation, a video image, a smartphone image, a fluoroscopy radiation dosage, an x-ray radiation dosage from an x-ray, in instrument removal from a holder, an instrument placement into a holder, an electrocautery dosage from an electrocautery device, an implant time at which an implant is placed in the patient, an audio recording, or an image.

32. The method of claim 31, wherein said graph comprises an interactive data file in which a user can identify a structure of the graph and view additional detail of the structure.

33. The method of claim 1, wherein the processor comprises a processor system.

34-200. (canceled)
